SEQUENCE LISTING (4 sequences)

SEQ ID NO: 1 - DNA, 20 bp, Artificial sequence
  Synthetic nucleotide
cgtggagctcagcggaacta

SEQ ID NO: 2 - DNA, 20 bp, Artificial sequence
  Synthetic nucleotide
aaccccagaaatttactccg

SEQ ID NO: 3 - DNA, 20 bp, Artificial sequence
  Synthetic nucleotide
gatggcaaggagaagtatag

SEQ ID NO: 4 - DNA, 20 bp, Artificial sequence
  Synthetic nucleotide
cagcggctggcgatcggccg

(19) United States
(12) Patent Application Publication
(10) Pub. No.: US 2018/0092894 A1
(43) Pub. Date: Apr. 5, 2018

(54) TREATMENT OF EOSINOPHILIC INFLAMMATORY DISEASE

(71) Applicant: Northwestern University, Evanston, IL (US)

(72) Inventors: Robert C. Kern, Chicago, IL (US); Jin-Young Min, Chicago, IL (US); Robert P. Schleimer, Chicago, IL (US); Bruce K. Tan, Chicago, IL (US)

(21) Appl. No.: 15/698,198

(22) Filed: Sep. 7, 2017

Related U.S. Application Data
(60) Provisional application No. 62/384,508, filed on Sep. 7, 2016.

(51) Int. Cl. A61K 31/4439; A61K 31/437; C12N 15/113; C12N 15/11; A61K 38/46; A61K 45/06; A61P 37/00; A61P 1/04; A61P 11/00

(52) U.S. Cl. CPC A61K 31/4439; A61K 31/437; C12N 15/1137; C12N 15/11; A61K 38/465; C12N 2310/20; A61P 37/00; A61P 1/04; A61P 11/00; C12N 2310/14; A61K 45/06

(57) ABSTRACT

Provided herein are compositions and methods for the treatment of eosinophilic inflammatory diseases of the mucosal surfaces using proton pump inhibitors. In particular, administration of inhibitors of H,K-ATPase (ATP12A) provides treatment for eosinophilic inflammatory diseases.

### CROSS-REFERENCE TO RELATED APPLICATION

The present application claims priority to U.S. Provisional Application 62/384,508, filed Sep. 7, 2016, which is herein incorporated by reference in its entirety.

### STATEMENT REGARDING FEDERAL FUNDING

This invention was made with government support under K23 DC012067 awarded by the National Institutes of Health. The government has certain rights in the invention.

### FIELD

Provided herein are compositions and methods for the treatment of eosinophilic inflammatory diseases of the mucosal surfaces using proton pump inhibitors (PPIs). In particular, administration of inhibitors of H,K-ATPase (ATP12A) provides treatment for eosinophilic inflammatory diseases.

### BACKGROUND

Chronic rhinosinusitis (CRS) with nasal polyps (CRSwNP), asthma, eosinophilic esophagitis and atopic dermatitis are diseases characterized by tissue eosinophilia where eosinophilia is associated with poor prognosis.

### SUMMARY

Provided herein are compositions and methods for the treatment of eosinophilic inflammatory diseases of the mucosal surfaces using proton pump inhibitors. In particular, administration of inhibitors of H,K-ATPase (ATP12A) provides treatment for eosinophilic inflammatory diseases.

Experiments conducted during development of embodiments herein demonstrate that PPIs reduce IL-13-stimulated eotaxin-3 expression by airway epithelial cells in vitro and are associated with lower in vivo levels in CRS tissue. Experiments further demonstrate that the non-gastric H,K-ATPase is involved in this response, identifying it as a target for treatment of CRSwNP. In particular, experiments demonstrated that tissue levels of type-2 inflammatory mediators, including IL-13, eotaxin-2, and eotaxin-3, were correlated with tissue eosinophilia and radiographic severity in CRS; eotaxin-3, the most highly induced eotaxin following IL-13 stimulation in human airway epithelial cells, was inhibited by PPIs in vitro, and lower in vivo levels of eotaxin-3 were observed in CRS patients taking PPIs compared with those without PPIs; and the inhibitory effect of PPIs in vitro occurred via multiple mechanisms, including inhibition of ngH,K-ATPase activity.

In some embodiments, provided herein are methods of treating or ameliorating the symptoms of an eosinophilic inflammatory disease in a subject comprising inhibiting ATP12A expression and/or activity within the subject. In some embodiments, the eosinophilic inflammatory disease is selected from the group consisting of asthma, atopic dermatitis, eosinophilic esophagitis, chronic rhinosinusitis with nasal polyps (CRSwNP). In some embodiments, inhibiting ATP12A expression and/or activity results in blunted IL-13-induction of eotaxin-3 mRNA, reduction in IL-13-induced epithelial cell production of eosinophil chemokines, suppression of the pathogenic effects of IL-4 and -13, and/or normalization of pH changes driven by type-2 cytokines. In some embodiments, inhibiting ATP12A expression and/or activity comprises inhibiting expression of ATP12A. In some embodiments, ATP12A expression is inhibited by inducing antisense inhibition, RNA interference, and or CRISPR/Cas. In some embodiments, inhibiting ATP12A expression and/or activity comprises administering to the subject an inhibitor of ATP12A activity. In some embodiments, the inhibitor is ATP12A specific. In some embodiments, the inhibitor is a general H⁺/K⁺-ATPase inhibitor. In some embodiments, the inhibitor is a small molecule, peptide, antibody, or antibody fragment. In some embodiments, the inhibitor is a substituted benzimidazole compound. In some embodiments, the substituted benzimidazole compound is selected from omeprazole, lansoprazole, dexlansoprazole, esomeprazole, pantoprazole, rabeprazole, and ilaprazole. In some embodiments, the inhibitor is co-administered with an additional agent for treating or ameliorating the symptoms of an eosinophilic inflammatory disease. In some embodiments, the inhibitor and additional agent are administered concurrently. In some embodiments, the inhibitor and additional agent are co-formulated. In some embodiments, the inhibitor and additional agent are administered serially. In some embodiments, the inhibitor is formulated for topical or local delivery to the mucosal or cutaneous surface being treated.

In some embodiments, provided herein are pharmaceutical compositions comprising (a) an inhibitor of ATP12A expression and/or activity, (b) an additional agent for treating or ameliorating the symptoms of an eosinophilic inflammatory disease, and (c) a pharmaceutically-acceptable carrier.

### BRIEF DESCRIPTION OF THE DRAWINGS

FIGS. 1A-1D. Increased levels of type-2 inflammatory mediators in nasal tissues and secretions of CRSwNP. Protein levels of IL-13 (FIG. 1A), eotaxin-2 (FIG. 1B), eotaxin-3 (FIG. 1C), and ECP (FIG. 1D) were measured in UT, nasal polyp, and nasal lavage fluid. Dot plots illustrate individual data points, and solid lines represent median with interquartile range.

FIGS. 2A-2D. IL-13-induced eotaxins protein secretion and inhibitory effects of omeprazole in airway epithelial cells. FIG. 2A, BEAS-2Bs and FIG. 2B, HNECs were stimulated for 48 h with IL-13. FIG. 2C, BEAS-2Bs and FIG. 2D, HNECs were pretreated with omeprazole for 2 h and stimulated for 48 h with IL-13. Eotaxins (FIG. 2A and FIG. 2B) and eotaxin-3 (FIG. 2C and FIG. 2D) levels in supernatants were measured by using ELISA.

FIGS. 3A-3B. Eotaxin-2 and eotaxin-3 levels were decreased in CRS patients taking PPIs at the time of sinus surgery. Protein levels of eotaxin-2 (FIG. 3A) and eotaxin-3 (FIG. 3B) in UT of CRS patients taking PPIs and those without PPIs were measured by using Luminex. Dot plots illustrate individual data points, and solid lines represent median with interquartile range.

FIGS. 4A-4C. H,K-ATPase inhibitors decreased IL-13-induced eotaxin-3 protein secretion. FIG. 4A, BEAS-2Bs were pretreated for 2 h with PPIs followed by IL-13 stimulation for 48 h. Eotaxin-3 levels in supernatants were measured by using ELISA. FIG. 4B, Correlations between the measured IC50 of PPIs for IL-13-induced eotaxin-3 with published ED50 of PPIs for gastric pH⁴². FIG. 4C, SCH-28080 was used with the same protocol as FIG. 4A.

FIGS. 5A-5D. IL-13-induced responses are mediated by the ngH,K-ATPase. FIG. 5A, After 6 h IL-13 stimulation with omeprazole or vehicle in BEAS-2Bs, fluorescence intensity was measured in confocal microscopic images (60× objective). FIG. 5B, Time course changes in fluorescence intensity in omeprazole- or vehicle-pretreated BEAS-2Bs after IL-13 stimulation. IL-13-induced eotaxin-3 mRNA expression was measured in FIG. 5C, BEAS-2Bs cultured in various [K⁺]ₑ-containing solution, and FIG. 5D, ATP12A or non-targeting siRNA-transfected HNECs.

FIGS. 6A-6D. Effects of omeprazole on IL-13-induced STAT6 phosphorylation and eotaxin-3 mRNA stability. FIG. 6A, In IL-13-stimulated BEAS-2Bs with omeprazole or vehicle, pSTAT6 and total STAT6 protein expression were measured by using Western blots. FIG. 6B, Semi-quantitative densitometry data for A (Mean±SEM, n=3-6). FIG. 6C, Experimental protocol for eotaxin-3 mRNA stability assessment using real-time PCR. FIG. 6D, Relative eotaxin-3 mRNA expression levels following treatment with actinomycin D and/or omeprazole.

FIG. 7. Model of role of the ngH,K-ATPase in facilitating inhibitory effects of PPIs on IL-13-medicated eotaxin-3 expression. In addition to the canonical IL-13/STAT6 pathway, IL-13-mediated eotaxin-3 expression may be affected by the ngH,K-ATPase activity. The ngH,K-ATPase is blocked by PPIs and other inhibitors including SCH-28080, ATP12A siRNA, and [K⁺]ₑ-free solution, resulting in H⁺,K⁺-flux and pHᵢ changes, which may affect expression of IL-13-mediated eotaxin-3.

FIGS. 8A-8D. IL-13-induced eotaxin-1, eotaxin-2, and eotaxin-3 gene expression in cultured airway epithelial cells and dose-dependent inhibition of IL-13-induced eotaxin-3 expression by omeprazole. FIG. 8A, BEAS-2Bs, and FIG. 8B, HNECs were stimulated for 48 h with IL-13 at escalating doses. FIG. 8C, BEAS-2Bs, and FIG. 8D, HNECs were pretreated with omeprazole for 2 h and stimulated for 48 h with IL-13 (5 ng/ml). Eotaxins (FIG. 8A and FIG. 8B) or eotaxin-3 (FIG. 8C and FIG. 8D) mRNA expression levels in total RNA from whole cells extracts were measured by using real-time PCR. Data represent means±SEMs compared with unstimulated cells (FIG. 8A and FIG. 8B) or vehicle-treated/IL-13-stimulated cells (FIG. 8C and FIG. 8D).

FIG. 9. Proton pump inhibitors did not inhibit mRNA expression of IFN-γ-induced CXCL-10 (IP-10), TNF-α-induced eotaxin-1, and IL-17-induced CXCL-1 in BEAS-2B cells. Cells were pretreated with various PPIs for 2 h and stimulated for 6 h with IFN-γ (10 ng/ml), TNF (100 ng/ml) or IL-17 (50 ng/ml). IFN-γ-induced CXCL10 (IP-10), TNF-α-induced eotaxin-1, and IL-17-induced CXCL1 mRNA expression levels in total RNA from cells were measured by using real-time PCR. Data represent means±SEM compared with vehicle-treated/cytokine-stimulated cells. O, omeprazole; L, lansoprazole; R, rabeprazole; P, pantoprazole; E, esomeprazole all at 5 μM

FIG. 10. Representative Western blot films of ATP12A protein (ngH,K-ATPase) expression in BEAS-2B cells, HNECs, and KNRK cells (positive control).

FIG. 11. SCH-28080 blocked IL-13-induced intracellular alkalization in BEAS-2B cells. Cells were pretreated with SCH-28080 or vehicle for 2 h prior to pHrodo® Green dye staining. After staining, cells were stimulated by IL-13 at 5 ng/ml. Fluorescence intensity were measured at various times before and after IL-13 stimulation up to 1 h using spectrofluorometry.

FIG. 12. Overall knockdown efficiencies of ATP12A mRNA and protein expression in HNECs. Cells were transfected with 25 pmol ON-TARGETplus ATP12A siRNA or non-targeting siRNA for 96 h. ATP12A mRNA expression levels in total RNA from cells were measured by using real-time PCR. Representative Western blot films of ATP12A protein expression in HNECs. Level of ATP12A mRNA was expressed as a percent of no siRNA transfected value.

FIG. 13. DNA templates for guide RNA (gRNA) targeting ATP12A (left). Knockout of ATP12A protein expression in BEAS-2B by CRISPR/cas9 technology (right). ATP12A was knocked out in the BEAS2B cell line using a lentivirus with targeting guide RNA-2 using the CRISPR-Cas9 technique. The synthetic gRNAs templates (CRISPR crRNA, from IDT)) was delivered using the transient tranfection reagent TransIT-X2 (Minis Bio, Madison, Wis.) into BEAS2B cells stably expressing the CAS9 protein (S. pyogenes CRISPR-Cas9). A clone was identified that completely knocked out ATP12A using Western blot (clones 1-13, 2-24).

FIG. 14. Complete inhibition of ATP12A using a CRISPR-CAS9 technology eliminated IL-13 induced eotaxin-3 gene expression in the BEAS-2B cell line. Untreated BEAS-2B, ATP12A wild-type cells and ATP12A knockout cells were stimulated with 5 ng/ml of IL-13 in the presence or absence of omeprazole. Eotaxin-3 protein secretion was measured in supernatants using ELISA. Data represent means±SEMs of 3 independent experiments. *P<0.05, **P<0.01, and ***P<0.001.

FIG. 15. IL-13-induced Periostin gene expression in cultured airway epithelial cells and inhibition of IL-13-induced Periostin expression by omeprazole (OME). Primary human nasal epithelial cells (NECs) were pretreated with omeprazole for 2 hours and stimulated for 48 hours with IL-13 (5 ng/mL). mRNA expression levels in total RNA from whole-cell extracts were measured by using real-time PCR. Data represent means±SEMs of 6 independent experiments. *P<0.05

FIG. 16. H,K-ATPase inhibitor decreased protein secretion of IL-13-induced mediators. Submerged cultured primary human nasal epithelial cells (NECs, n=3) were treated with 5 ng/ml of IL-13 with or without pretreatment for 2 h with acid-activated omeprazole (5 μM). After 6 hours of stimulation, the cells were harvested and mRNA levels of several candidate genes including eotaxin-3 (CCL26), periostin (POSTN), arachidonate 15-lipoxygenase (ALOX15) and claudin-5 (CLDN5) were determined by RNA-Seq. Gene expression data represent the fragments per kilobase mapped (fpkm).

FIG. 17. IL-13-induced MUC5AC gene expression and the effect of H,K-ATPase inhibitor on IL-13-induced MUC5AC. Primary human nasal epithelial cells (NECs) were grown in transwells at air-liquid interface to induce differentiation of epithelium into ciliated differentiated epithelium. 5 ng/ml of basal IL-13 was then applied and gene expression of mucin 5AC (MUC5AC) was measured at 24 hrs after stimulation with or without pretreatment for 2 h with acid-activated omeprazole (5 μM). Data represent means±SEMs of 8 independent experiments. **P<0.01

FIG. 18. IL-13 acidifies airway surface liquid pH that is reversed by inhibition of the non-gastric H+/K+ATPase. Primary human nasal epithelial cells (NECs) were grown at air-liquid interface to induce differentiation of epithelium into ciliated differentiated epithelium. When confluent, the cell culture was replaced with unbuffered live cell imaging solution (LCIS). 5 ng/ml of basally applied IL-13 was then applied with or without pretreatment for 1 hr with acid-activated omeprazole (5 μM). pH measurements were made by applying 50 ml of a SNARF-dextran ratiometric pH dye and read on a spectrofluorometer. Data represent means±SEMs of 11 independent experiments. *P<0.05, **P<0.01

FIG. 19. The pH of nasal secretions from control and chronic rhinosinusitis with nasal polyps (CRSwNP) patients. Nasal secretions from the middle meatus were collected by using an endoscopically placed 0.375-inch polyvinyl alcohol sponge that was inserted between the middle turbinate and the adjacent uncinate process for 10 minutes before removal from control and CRS patients. The pH of collected nasal secretions were measured using a micro-pH meter. *P<0.05

FIG. 20. Airway pH was significantly negatively correlated with type-2 cytokines. Level of type 2 cytokines in nasal tissue from the same patients whose nasal secretion was collected were assessed by Luminex assay. Correlations between nasal pH and the levels of the type 2 cytokines (IL-13 and IL-4) were assessed by using a Spearman rank correlation test. *P<0.05 and **P<0.01.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an ATP12A inhibitor” is a reference to one or more ATP12A inhibitors and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “pharmaceutically acceptable carrier” refers to non-toxic solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed, and is compatible with other ingredients of the formulation. The pharmaceutically acceptable carrier is appropriate for the formulation employed. For example, if the therapeutic agent is to be administered orally, the carrier may be a gel capsule. A “pharmaceutical composition” typically comprises at least one active agent (e.g., PA nanostructures) and a pharmaceutically acceptable carrier.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions herein) to a subject or in vivo, in vitro, or ex vivo cells, tissues, and organs. Exemplary routes of administration to the human body can be through the eyes (e.g., intraocularly, intravitrealy, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc. As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to binding other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope. Some embodiments herein comprise generating and/or administering antibodies that bind and inhibit ATP12A.

As used herein, the term “antibody fragment” refers to a portion of a full-length antibody, including at least a portion of an antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen. Other antibody fragments will be understood by skilled artisans. Some embodiments herein comprise generating and/or administering antibody fragments that bind and inhibit ATP12A.

### DETAILED DESCRIPTION

Provided herein are compositions and methods for the treatment of eosinophilic inflammatory diseases of the mucosal surfaces using proton pump inhibitors. In particular, administration of inhibitors of H,K-ATPase (ATP12A) provides treatment for eosinophilic inflammatory diseases.

Chronic rhinosinusitis (CRS) is characterized by local inflammation of the sinonasal mucosa with symptoms persisting for at least 12 weeks (ref 1; incorporated by reference in its entirety). It is further classified into 2 clinical phenotypes: CRS with nasal polyps (CRSwNP) and CRS without nasal polyps (CRSsNP) (refs. 1-3; incorporated by reference in their entireties). In Western populations, CRSwNP is frequently associated with type-2 inflammation and tissue eosinophilia (ref 4; incorporated by reference in its entirety). Since tissue eosinophilia has been implicated in increased post-surgical recurrence rates (refs. 5-6; incorporated by reference in their entireties) and decreased improvements in quality of life outcomes (ref. 7; incorporated by reference in its entirety) strategies for blocking eosinophil recruitment are desirable for treatment of CRSwNP.

ATPase, H⁺/K⁺ transporting, nongastric, alpha polypeptide (also known as ATP12A, ngH,K-ATPase, etc.) is a protein that in humans is encoded by the ATP12A gene (Sverdlov et al. Genomics. 32 (3): 317-27; Yang-Feng et al. Genomics. 2 (2): 128-38; incorporated by reference in their entireties). ATP12A belongs to the family of P-type cation transport ATPases. This gene encodes a catalytic subunit of the ouabain-sensitive H⁺/K⁺-ATPase that catalyzes the hydrolysis of ATP coupled with the exchange of H⁺ and K⁺ ions across the plasma membrane. It is also responsible for potassium absorption in various tissues.

Experiments were conducted during development of embodiments herein to assess the effect of type-2 mediators (e.g., IL-13 and eotaxin-3) on tissue eosinophilia and disease severity in CRS. Further investigation focused on PPI suppression of eotaxin-3 expression in vivo and in vitro with exploration of underlying mechanisms. Type-2 mediator levels in nasal tissues and secretions were measured by multiplex immunoassay. Eotaxin-3 and other chemokines expressed in IL-13-stimulated human sinonasal epithelial cells (HNECs) and BEAS-2Bs with or without PPIs were assessed by using ELISA, Western blot, real-time PCR, and intracellular pH (pHᵢ) imaging. Nasal tissues and secretions from CRSwNP patients had increased IL-13, eotaxin-2 and eotaxin-3 levels, and these were positively correlated with tissue ECP and radiographic scores in CRS. IL-13-stimulation of HNECs and BEAS-2Bs dominantly induced eotaxin-3 expression, which was significantly inhibited by PPIs. CRS patients taking PPIs also showed lower in vivo eotaxin-3 levels compared with those without PPIs. Using pHᵢ imaging and by altering extracellular [K⁺], it was found that IL-13 enhanced H⁺,K⁺-exchange, which was blocked by PPIs and the mechanistically unrelated H,K-ATPase inhibitor, SCH-28080. Furthermore, knockdown of ATP12A (gene for the non-gastric H,K-ATPase [ngH,K-ATPase]) significantly attenuated IL-13-induced eotaxin-3 expression in HNECs. PPIs also had effects on accelerating IL-13-induced eotaxin-3 mRNA decay. These results demonstrate that PPIs reduce IL-13-induced eotaxin-3 expression by airway epithelial cells. Furthermore, mechanistic studies indicate the heretofore unknown and unexpected conclusion that the ngH,K-ATPase is necessary for IL-13-mediated epithelial responses, and its inhibitors, including PPIs, are a therapy for CRSwNP, by reducing epithelial production of eotaxin-3.

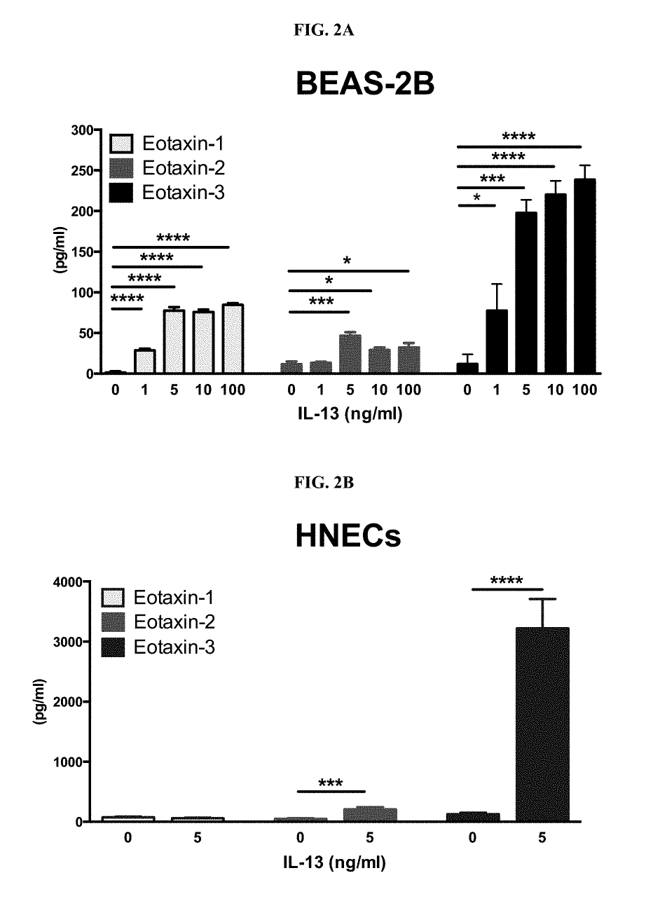

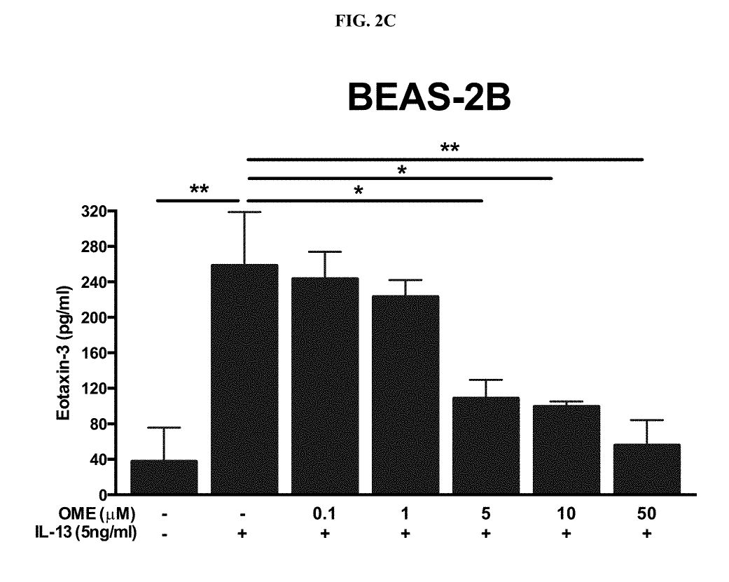

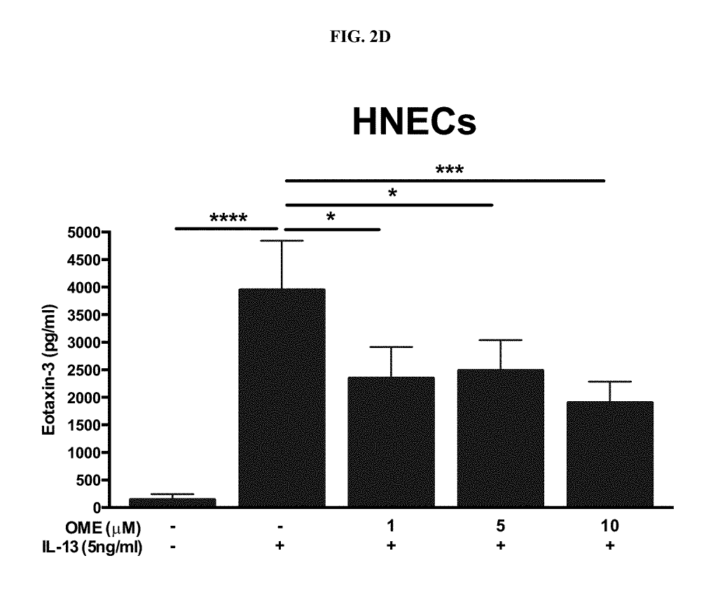

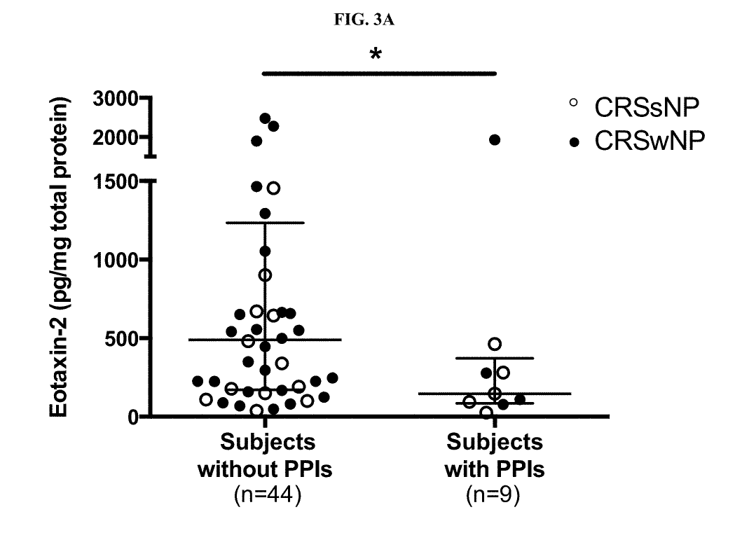

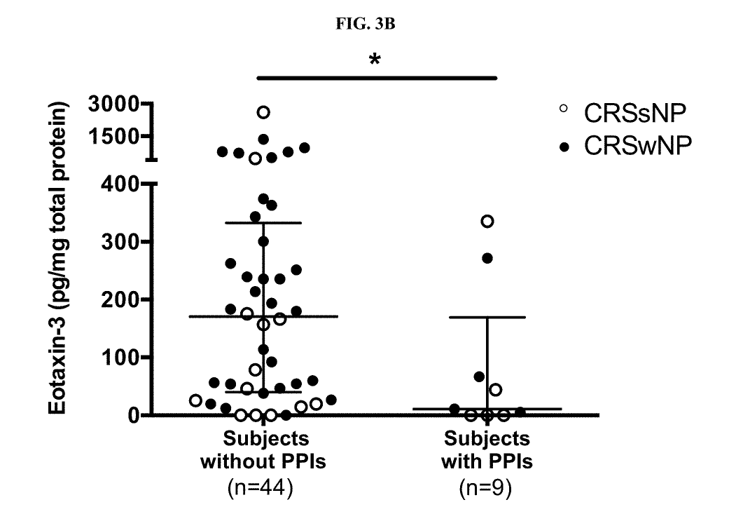

Experiments conducted during development of embodiments herein demonstrate that eotaxin-3 is a biomarker for tissue IL-13 levels, eosinophilia, and radiographic severity in CRS (Tables 1 and 2). In vitro profiles of the eotaxins by IL-13-stimulated HNECs and BEAS-2Bs were comprehensively evaluated, and it was found that both cell types, but particularly HNECs, predominantly expressed eotaxin-3 (FIG. 2). It was confirmed that PPIs had similar inhibitory effects on IL-13-induced eotaxin-3 expression by HNECs in vitro (FIG. 2), and that PPIs have similar effects on patients taking these medications (FIG. 3).

In vivo analysis demonstrates that eotaxin-2, -3 and IL-13 levels were intercorrelated in tissues and secretions, and positively correlated with tissue eosinophilia and radiographic severity in CRS (Tables 1). Additionally, it was found that the eotaxins could be measured in nasal secretions and significantly reflected tissue eosinophilia (Table 1) and IL-13 levels (Table 2), indicating their value as non-invasive biomarkers. Although these measures were increased in both UT and NP in CRSwNP, and were actually higher in NP, the significant correlations between mediators and radiographic and eosinophilic severity were only found within UT (Tables 1 and 5). This indicates that the extent of type-2 inflammation in UT may be more reliably representative of disease burden of CRS.

TABLE 1
Correlations between type-2 inflammatory mediators
and tissue eosinophilia or radiographic severity

| Type-2 | ECP in UT | | CT scores | |
|---|---|---|---|---|
| inflammatory | (Total Subjects*) | | (Patients with CRSwNP^{†}) | |
| mediators | r | P-value | r | P-value |

in UT

| | | | | |
|---|---|---|---|---|
| IL-13 | 0.84 | <0.0001 | 0.49 | 0.002 |
| Eotaxin-1 | 0.19 | 0.31 | 0.34 | 0.04 |
| Eotaxin-2 | 0.70 | <0.0001 | 0.60 | 0.0002 |
| Eotaxin-3 | 0.54 | 0.002 | 0.34 | 0.049 |
| ECP | — | — | 0.58 | 0.0003 |

in Nasal Lavage Fluid

| | | | | |
|---|---|---|---|---|
| IL-13 | 0.55 | 0.001 | 0.11 | 0.41 |
| Eotaxin-1 | 0.12 | 0.52 | 0.09 | 0.49 |
| Eotaxin-2 | 0.51 | 0.003 | 0.15 | 0.29 |
| Eotaxin-3 | 0.49 | 0.004 | 0.14 | 0.31 |
| ECP | 0.26 | 0.30 | 0.05 | 0.81 |

ECP, eosinophil cationic protein; UT, uncinate tissue; CT, computed tomography; CRSwNP, chronic rhinosinusitis with nasal polyps
*N = 32 for correlations between ECP in UT with measures in UT and nasal lavage fluid
^{†}N = 34 and 55 for correlations between CT scores with measures in UT and nasal lavage fluid respectively; UT tissue was not always available in instances of revision surgery.

TABLE 2
Correlation between type-2 inflammatory
mediators and IL-13 in nasal tissues

| | |
|---|---|
| Type-2 | IL-13 in UT |
| inflammatory | (Total Subjects, n = 32) |

| | | |
|---|---|---|
| mediators | R | P-value |

in UT

| | | |
|---|---|---|
| Eotaxin-1 | 0.39 | 0.03 |
| Eotaxin-2 | 0.79 | <0.0001 |
| Eotaxin-3 | 0.61 | <0.001 |
| ECP | 0.84 | <0.0001 |

in Nasal lavage fluid

| | | |
|---|---|---|
| Eotaxin-1 | 0.24 | 0.19 |
| Eotaxin-2 | 0.57 | <0.001 |
| Eotaxin-3 | 0.68 | <0.0001 |
| ECP | 0.25 | 0.32 |

TABLE 5
Correlations between type-2 inflammatory mediators in nasal
polyp and tissue eosinophilia or radiographic severity

| Type-2 | ECP in NP | | CT scores | |
|---|---|---|---|---|
| inflammatory | (Patients with CRSwNP*) | | (Patients with CRSwNP^{†}) | |
| mediators | r | P-value | r | P-value |

in NP

| | | | | |
|---|---|---|---|---|
| IL-13 | 0.30 | 0.22 | 0.09 | 0.56 |
| Eotaxin-1 | 0.08 | 0.75 | 0.12 | 0.43 |
| Eotaxin-2 | 0.13 | 0.62 | 0.08 | 0.61 |
| Eotaxin-3 | −0.35 | 0.16 | −0.06 | 0.69 |
| ECP | — | — | 0.34 | 0.03 |

Using in vitro experiments, it was found that eotaxin-3 was the predominant eotaxin produced by HNECs (FIGS. 2, A and B). While eotaxin-2 in vivo levels were highly elevated in CRSwNP tissue extracts, it was only modestly induced in IL-13-stimulated HNECs. This indicates that the majority of eotaxin-2 is attributable to non-epithelial inflammatory cells.

Safe systemic options for long-term medical management of CRSwNP are currently lacking. Although corticosteroids are the mainstay of medical management in CRSwNP, their effects are short lived and long-term treatment is limited by systemic side effects (refs. 28, 56-57; incorporated by reference in their entireties).

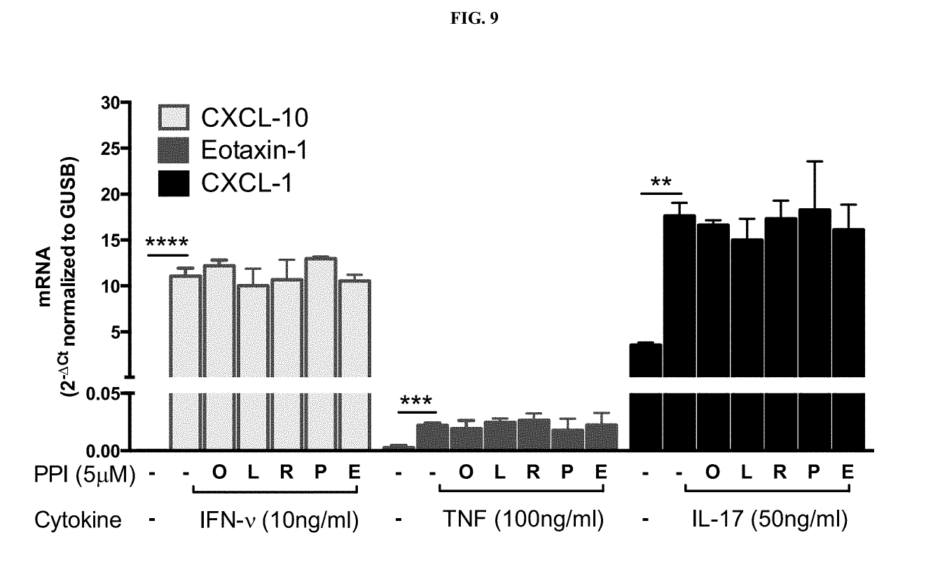

Experiments conducted during development of embodiments herein demonstrated that IL-13-induced eotaxin-3 protein secretion was reduced 57.9% in BEAS-2Bs and 37.1% in HNECs by 5 μM omeprazole (FIGS. 2, C and D) in vitro. Notably, these in vitro anti-inflammatory effects were specific to type-2 cytokine-mediated responses (FIG. 9). Furthermore, CRS patients who were taking PPIs at the time of surgery showed significantly lower levels of eotaxin-3 and eotaxin-2 in nasal tissue compared with patients not receiving PPIs (FIG. 3). These results show promise that our in vitro results might be replicated in vivo but further studies including clinical trials are needed to prospectively evaluate their efficacy in CRSwNP.

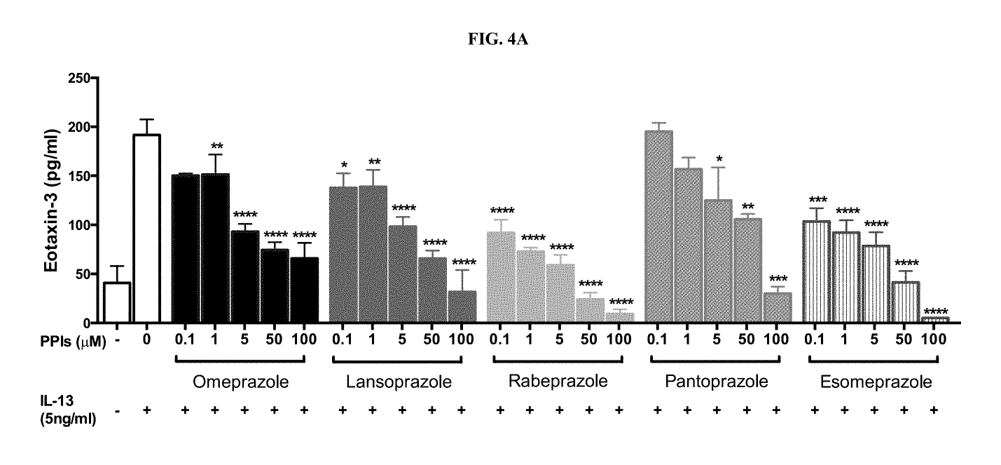

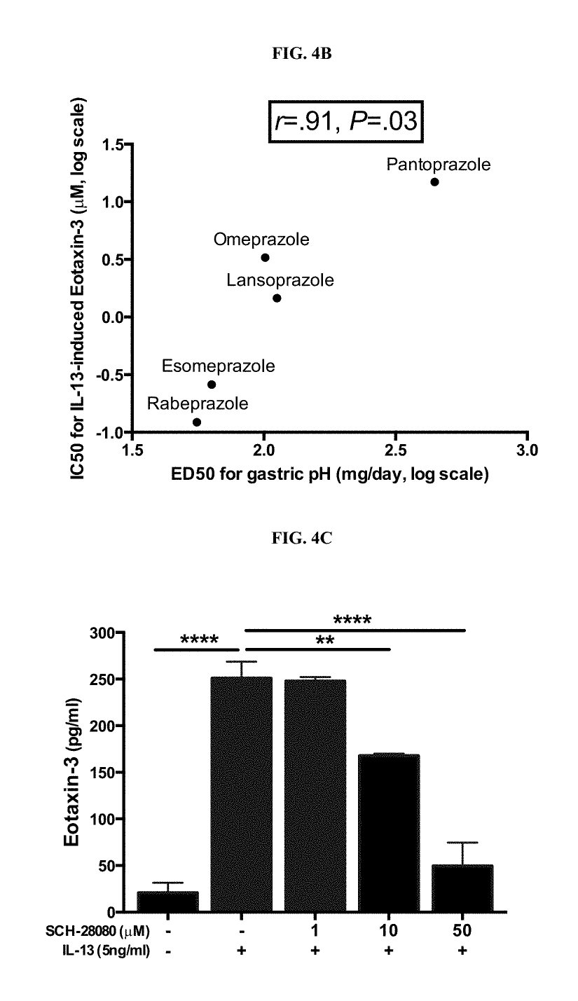

Experiments conducted during development of embodiments herein indicate that the mechanism by which PPIs inhibit IL-13-induced eotaxin-3 involves inhibition of ngH,K-ATPase activity. Specifically, PPIs inhibited IL-13-induced eotaxin-3 expression with the same rank order as inhibition of gastric acid secretion, indicating a near-perfect structure-activity relationships of PPIs for these two effects (FIG. 4,B) and further, IL-13-induced eotaxin-3 expression was suppressed by SCH-28080, a mechanistically distinct H,K-ATPase inhibitor (FIG. 4,C). Since the gH,K-ATPase, the known target of PPIs, is not expressed in airway epithelium, the data indicates that ngH,K-ATPase, the only other P-type ATPase with H⁺,K⁺-antiporting activity, is the source of activity. The ngH,K-ATPase shares approximately 65% sequence homology with the gH,K-ATPase and Na,K-ATPase, and is moderately sensitive to their inhibitors (refs. 44, 63-65; incorporated by reference in their entireties).

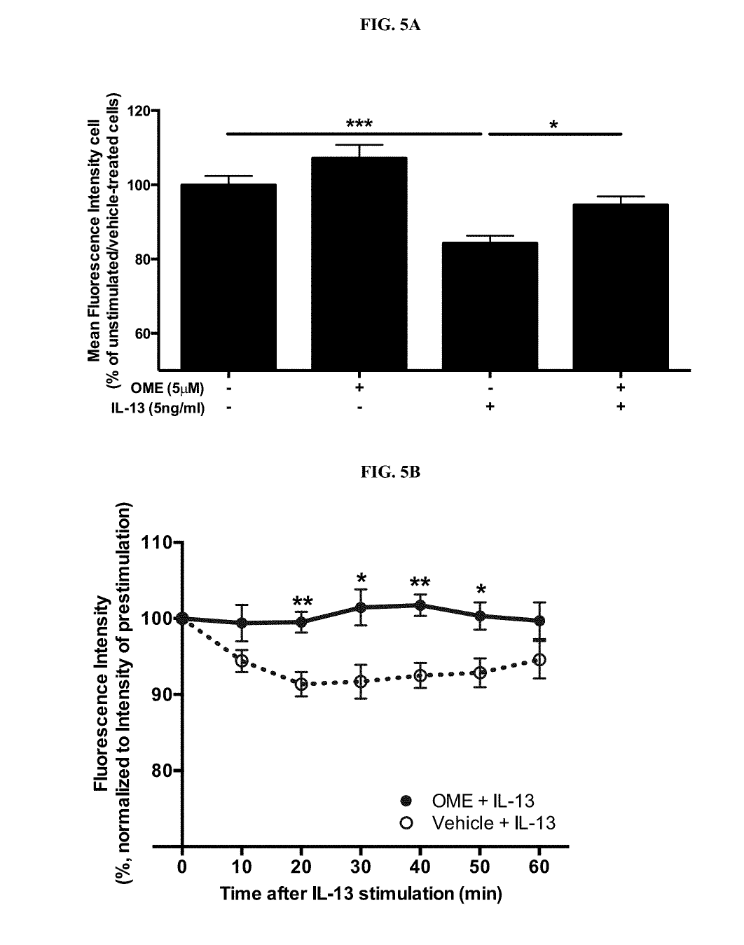

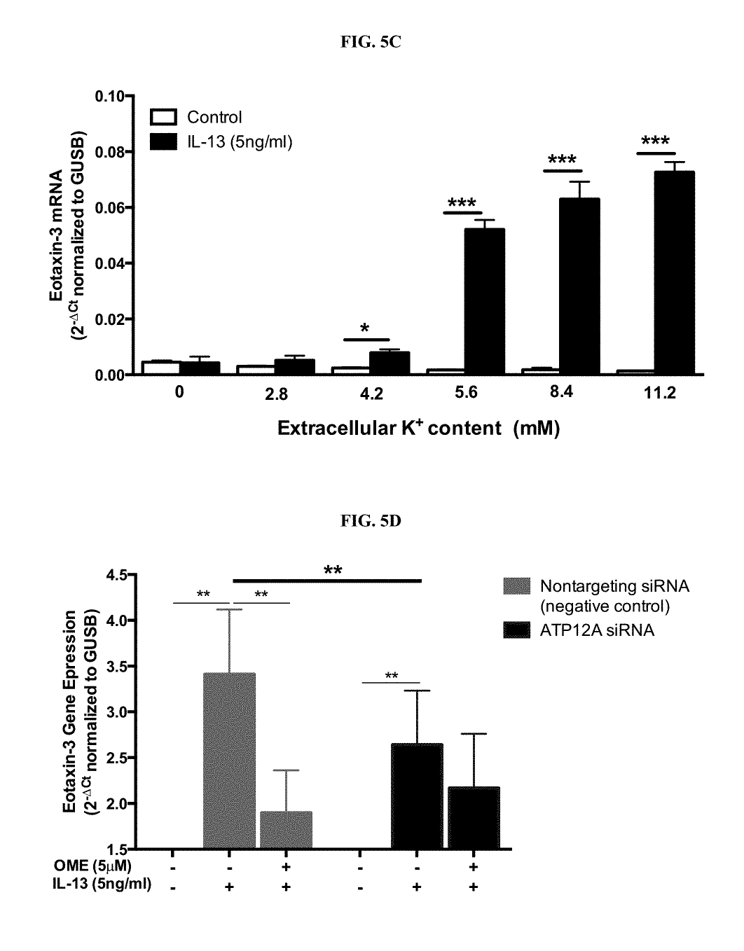

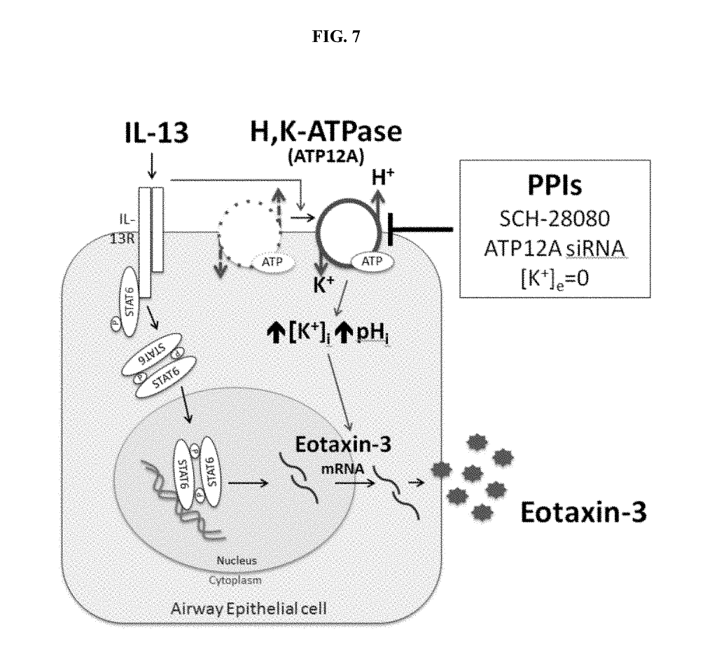

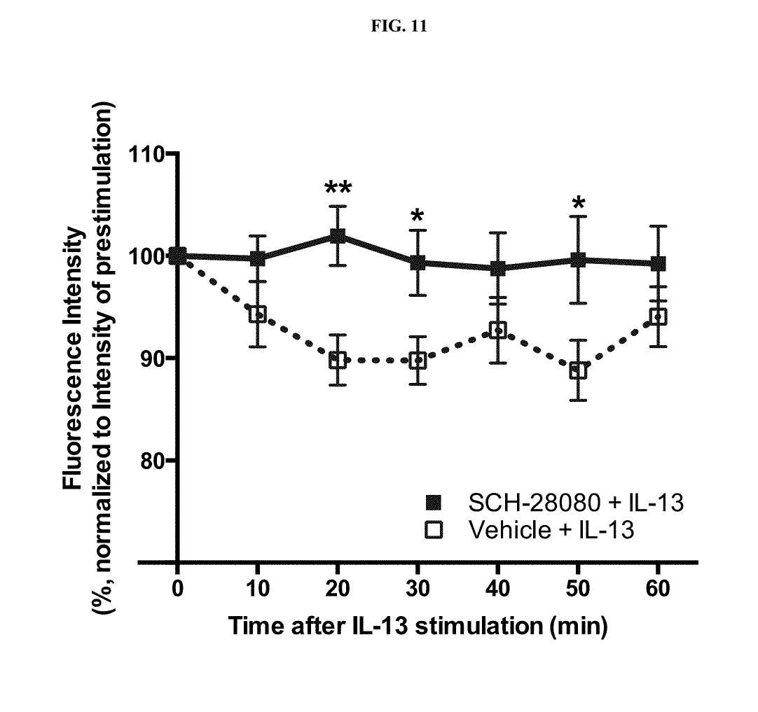

Experiments conducted during development of embodiments herein demonstrate a role for ngH,K-ATPase activity in optimal expression of IL-13-responsive genes, like eotaxin-3, might require (FIG. 7). This is supported by findings that IL-13 stimulation induced rapid intracellular alkalization, that was blocked by omeprazole (FIGS. 5, A and B) and SCH-28080 (FIG. 11); eotaxin-3 mRNA induction by IL-13 was highly sensitive to [K⁺]ₑ, and was completely eliminated in [K⁺]ₑ-free solution; and knockdown of ATP12A significantly blunted IL-13-induction of eotaxin-3 mRNA (FIG. 5, D).

Taken together, experiments conducted during development of embodiments herein demonstrate that inhibitors of the ngH,K-ATPase are of significant therapeutic value in the IL-13-mediated responses found in CRSwNP.

In some embodiments, inhibition (e.g., complete inhibition) of ATP12A function (e.g., by CRISPR) eliminates IL-13-induced epithelial cell production of eosinophil chemokines like CCL26. In some embodiments, such strategies to reduce ATP12A activity are of therapeutic value, and are provided in embodiments herein.

In some embodiments, in addition to suppression of eosinophil chemokine expression, the inhibition of the non-gastric H+/K+ ATPase in epithelial cells broadly suppresses the known pathogenic effects of IL-4 and -13, including epithelial barrier disruption, extracellular matrix reorganization and mucus hyper-production.

In some embodiments, airway surface liquid pH reflects the extent of IL-4 and -13 driven inflammation. Inhibitors of the non-gastric H+/K+ ATPase normalize the pH changes driven by type-2 cytokines.

Accordingly, in some embodiments, provided herein are methods of treating, preventing, and/or ameliorating the symptoms of eosinophilic inflammatory diseases of the mucosal surfaces by inhibition of the activity or expression of ATP12A. Diseases and conditions that are addressed (e.g., treated, prevented, ameliorated, etc.) in embodiments herein include, but are not limited to asthma, atopic dermatitis, eosinophilic esophagitis, eosinophilic gastrointestinal disease, chronic rhinosinusitis with nasal polyps (CRSwNP), etc. In some embodiments, an inhibitor (of the activity) of ATP12A is administered to a subject (e.g., by any suitable route of administration and within any suitable pharmaceutical formulation). In some embodiments, expression of ATP12A is inhibited (e.g., partially or completely), for example, by siRNA, or genetic manipulation (e.g., by CRISPR).

In some embodiments, methods herein comprise administering a proton pump inhibitor (PPI) to a subject at risk of an eosinophilic inflammatory disease and/or suffering from an eosinophilic inflammatory disease. PPIs act by irreversibly blocking an H⁺/K⁺ ATPase (e.g., ATP12A), or, more commonly, the gastric proton pump) of the gastric parietal cells.

In some embodiments, a PPI is a small molecule drug. Exemplary PPIs are already in medical use. In some embodiments, a PPI is a substituted benzimidazole compound. In some embodiments, a PPI for use in embodiments herein is selected from the group consisting of omeprazole (5- or 6-methoxy-2-{[(4-methoxy-3,5-dimethylpyridin-2-yl)methyl]sulfinyl}-1H-ben-zimidazole), lansoprazole (2-{[3-methyl-4-(2,2,2-trifluoroethoxyl)pyridin-2-yl]methylsulfinyl-1H-ben-zo(d)imidazole), dexlansoprazole, esomeprazole (S-5-methoxy-2-{(4-methoxy-3,5 dimethylpyridin-2-yl)methylsufinyl]-3H-benzoimidazole), pantoprazole (RS-6-(difluoromethoxy))-2-[(3,4-dimethoxypyridin-2-yl)methylsulfinyl]-1H-benzo(d)imidazole), rabeprazole 2-([4-(3-methoxypropoxy)-3-methylpyridin-2-yl]methylsulfinyl)-1H-benzo(d)-imidazole, and ilaprazole.

In some embodiments, other compositions for inhibiting the activity of ATP12A include peptide ATP12A inhibitors, antibody or antibody fragments, etc.

In some embodiments, the technology provides a method for inhibiting ATP12A activity by administering an antibody or fragment that recognizes, binds, and inhibits the activity of ATP12A. In some embodiments, the antibody is a monoclonal antibody and in some embodiments the antibody is a polyclonal antibody. In some embodiments, the antibody is, for example, a human, humanized, or chimeric antibody. Monoclonal antibodies against target antigens are produced by a variety of techniques including conventional monoclonal antibody methodologies such as the somatic cell hybridization techniques of Köhler and Milstein (Nature, 256:495 (1975)). Although in some embodiments, somatic cell hybridization procedures are preferred, other techniques for producing monoclonal antibodies are contemplated as well.

In some embodiments, methods herein comprise inhibiting the expression of ATP12A. Multiple methods of altering gene expression within a cell, tissue, or subject are known in the field (e.g., RNAi, antisense RNA, gene therapy, CRISPR, etc.).

In some embodiments, a nucleic acid is used to modulate expression of ATP12A.

For example, in some embodiments a small interfering RNA (siRNA) is designed to target and degrade ATP12A. siRNAs are double-stranded RNA molecules of 20-25 nucleotides in length. While not limited in their features, typically an siRNA is 21 nucleotides long and has 2-nt 3′ overhangs on both ends. Each strand has a 5′ phosphate group and a 3′ hydroxyl group. In vivo, this structure is the result of processing by Dicer, an enzyme that converts either long dsRNAs or small hairpin RNAs (shRNAs) into siRNAs. However, siRNAs can also be synthesized and exogenously introduced into cells to bring about the specific knockdown of a gene of interest. Essentially any gene of which the sequence is known can be targeted based on sequence complementarity with an appropriately tailored siRNA. For example, those of ordinary skill in the art can synthesize an siRNA (see, e.g., Elbashir, et al., Nature 411: 494 (2001); Elbashir, et al. Genes Dev 15:188 (2001); Tuschl T, et al., Genes Dev 13:3191 (1999); incorporated by reference in their entireties).

In some embodiments, RNAi is utilized to inhibit expression of ATP12A. In some embodiments, RNAi is used to modulate expression of ATP12A. RNAi represents an evolutionarily conserved cellular defense for controlling the expression of foreign genes in most eukaryotes, including humans. RNAi is typically triggered by double-stranded RNA (dsRNA) and causes sequence-specific degradation of single-stranded target RNAs (e.g., an mRNA). The mediators of mRNA degradation are small interfering RNAs (siRNAs), which are normally produced from long dsRNA by enzymatic cleavage in the cell. siRNAs are generally approximately twenty-one nucleotides in length (e.g. 21-23 nucleotides in length) and have a base-paired structure characterized by two-nucleotide 3′ overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, an RNase III enzyme (e.g., Dicer) converts the longer dsRNA into 21-23 nt double-stranded siRNA fragments. In some embodiments, RNAi oligonucleotides are designed to target the junction region of fusion proteins. Chemically synthesized siRNAs have become powerful reagents for genome-wide analysis of mammalian gene function in cultured somatic cells. Beyond their value for validation of gene function, siRNAs also hold great potential as gene-specific therapeutic agents (see, e.g., Tuschl and Borkhardt, Molecular Intervent. 2002; 2(3): 158-67, herein incorporated by reference).

In other embodiments, shRNA techniques (See e.g., 20080025958, herein incorporated by reference in its entirety) are utilized to modulate (e.g., inhibit) expression of ATP12A. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA that is bound to it. shRNA is transcribed by RNA polymerase III.

In some embodiments, an antisense nucleic acid (e.g., an antisense DNA oligo, an antisense RNA oligo) is used to modulate the expression of ATP12A. For example, in some embodiments, expression of ATP12A is inhibited using antisense compounds that specifically hybridize with nucleic acids ATP12A. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation (e.g., inhibition) of the expression of ATP12A.

As an alternative (or in addition to) the methods of inhibiting expression above, in some embodiments, nucleic acids are employed to inhibit ATP12A activity, For example, one of ordinary skill in the art can design and produce RNA aptamers or other nucleic acids that specifically recognize and bind to ATP12A, for instance by using SELEX or other in vitro evolution methods known in the art.

In some embodiments, ATP12A activity is inhibited by specifically degrading or inducing an altered conformation of oATP12A such that it is less effective. In some embodiments, an inhibitor is a “designed ankyrin repeat protein” (DARPin) (see, e.g., Stumpp M T & Amstutz P, “DARPins: a true alternative to antibodies”, Curr Opin Drug Discov Devel 2007, 10(2): 153-59, incorporated herein in its entirety for all purposes).

In some embodiments, ATP12A activity and/or expression are inhibited using the CRISPR/Cas system. “CRISPRs” (clustered regularly interspaced short palindromic repeats), as described herein, are segments of prokaryotic DNA containing short repetitions of base sequences. Each repetition is followed by short segments of “spacer DNA” from previous exposures to a bacterial virus or plasmid. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. CRISPR/Cas system has been used for gene editing (adding, disrupting or changing the sequence of specific genes) and gene regulation in species throughout the tree of life. By delivering the Cas9 protein and appropriate guide RNAs into a cell, the organism's genome can be cut at any desired location. One can use CRISPR to build RNA-guided gene editing tools capable of altering the genome of a subject. In some embodiments, the CRISPR/Cas system is utilized to inhibit (e.g., partially or completely) the expression of ATP12A in a subject, tissue, or cells. In some embodiments, the CRISPR/Cas system is utilized to produce ATP12A that is of reduced activity (in a subject, tissue, or cells.

In some embodiments, the therapies and therapeutic compositions a described herein are employed with one or more co-therapies or co-therapeutics for the treatment of eosinophilia and/or CRS, and/or for addressing symptoms of eosinophilia and/or CRS. In some embodiments, one or more therapies and/or therapeutics are co-administered with the therapies and therapeutic compositions described herein. In some embodiments, co-therapies and/or co-therapeutics are administered with or without (known) synergism.

In some embodiments, co-therapies/co-therapeutics are provided for the treatment of CRS (e.g., CRSwNP) are provided. In some embodiments, co-therapies/co-therapeutics for use with the compositions and methods described herein include systemic and topical bactericidal or fungicidal drugs that have been demonstrated to exhibit very good short-term efficacy for reduction of microbial density in the paranasal mucosa and concomitant alleviation of CRS clinical symptoms (See, e.g., Kaplan (2013) Can Fam Physician 59(12):1275-1281; Lim et al. (2008) Am J Rhinol 22(4):381-389; Huang and Govindaraj (2013) Curr Opin Otolaryngol Head Neck Surg 21(1):31-38; incorporated by reference in their entireties) Long-term use of antibiotics is not recommended, however, due to concerns over the danger of promoting expansion of resistant bacteria (Kennedy and Borish (2013) Am J Rhinol Allergy 27(6):467-472; incorporated by reference in its entirety). In some embodiments, co-therapies/co-therapeutics include various probiotic agents and other “microbiome rebalancing” strategies (Cleland et al. (2014) Int Forum Allergy Rhinol 4(4):309-314; Mukerji et al. (2009) Otolaryngol Head Neck Surg 140(2):202-208; incorporated by reference in their entireties)). Other co-therapies that may find use in embodiments herein include intranasal irrigations with colloidal silver, surfactant solutions, sodium hyaluronate, methylglyoxal, xylitol solution, and isotonic or hypertonic saline (Goggin et al. (2014) Int Forum Allergy Rhinol 4(3):171-175; Chiu et al. (2008) Am J Rhinol 22(1):34-37; Casale et al. (2014) Am J Rhinol Allergy 28(4):345-348; Kilty et al. (2011) Int Forum Allergy Rhinol 1(5):348-350; Weissman et al. (2011) Laryngoscope 121(11):2468-72; van den Berg et al. (2014) Otolaryngol Head Neck Surg 150(1):16-21; Ural et al. (2009) J Laryngol Otol 123(5):517-21; incorporated by reference in their entireties)) Ultrasound treatment to disrupt bacterial biofilm may also find use as a co-therapy herein (Ansari et al. (2012) Physiother Theory Pract 28(2):85-94; Young et al. (2010) J Laryngol Otol 124(5):495-499; incorporated by reference in their entireties).

In some embodiments, co-therapies/co-therapeutics are provided for the treatment of asthma are provided. In some embodiments, co-therapies/co-therapeutics for use with the compositions and methods described herein include inhaled corticosteroids, cromolyn, omalizumab, inhaled long-acting beta2-agonists, leukotriene modifiers, theophylline, short-acting beta2-agonists, etc.

In some embodiments, co-therapies/co-therapeutics are provided for the treatment of atopic dermatitis are provided. In some embodiments, co-therapies/co-therapeutics for use with the compositions and methods described herein include topical corticosteroids or oral corticosteroids, UV light treatment, cyclosporine, interferon, antihistamine, etc.

In some embodiments, co-therapies/co-therapeutics are provided for the treatment of eosinophilic esophagitis are provided. In some embodiments, co-therapies/co-therapeutics for use with the compositions and methods described herein include elimination diet, acid suppression, topical glucocorticoids, esophageal dilation, systemic glucocorticoids, antihistamines, immunosuppressants, and immunomodulators.

Does, routes of administration, and formulations (e.g., separate from the ATP12A inhibitors herein, co-formulated with the ATP12A inhibitors herein) of co-therapeutics are understood in the field.

In some embodiments, provided herein are pharmaceutical compositions comprising an inhibitor of ATP12A activity or expression, alone or in combination with at least one other agent, such as a stabilizing compound, and may be administered in any sterile, biocompatible pharmaceutical carrier, including, but not limited to, saline, buffered saline, dextrose, and water.

As is well known in the medical arts, dosages for any one patient depends upon many factors, including the patient's size, body surface area, age, the particular compound to be administered, sex, time and route of administration, general health, and interaction with other drugs being concurrently administered.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, topical, or intranasal administration.

For injection, pharmaceutical compositions may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions are formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

In some embodiments, inhibitors of ATP12A activity or expression may be formulated for delivery by inhalation. As used herein, the term “aerosol” is used in its conventional sense as referring to very fine liquid or solid particles carries by a propellant gas under pressure to a site of therapeutic application. In some embodiments, liquid formulation of inhibitors of ATP12A activity or expression is used with a pharmaceutically acceptable carrier in flowable liquid form. Such formulations, when used for delivery, are generally solutions, e.g. aqueous solutions, ethanolic solutions, aqueous/ethanolic solutions, saline solutions and colloidal suspensions.

Pharmaceutical compositions include compositions wherein the active ingredients are contained in an effective amount to achieve the intended purpose. Determination of effective amounts is well within the capability of those skilled in the art, especially in light of the disclosure provided herein.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Pharmaceutical preparations for oral administration include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising an inhibitor of ATP12A formulated in a pharmaceutical acceptable carrier may be prepared, placed in an appropriate container, and labeled for treatment of an indicated condition.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

In some embodiments, a therapeutically effective dose may be estimated initially from assays and/or animal models. A therapeutically effective dose refers to that amount that ameliorates symptoms of the disease state or unwanted condition. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. Data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration. The exact dosage is chosen by the individual clinician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination (s), reaction sensitivities, and tolerance/response to therapy. Long acting pharmaceutical compositions might be administered every 3 to 4 days, every week, or once every two weeks depending on half-life and clearance rate of the particular formulation. Typical dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; 5,225,212; WO2004/097009, or WO2005/075465, each of which are herein incorporated by reference).

### EXPERIMENTAL

### Materials and Methods

### Subjects and Sample Collection

Healthy controls and patients with CRS (refs. 2, 34; incorporated by reference in their entireties) were recruited from the Otolaryngology and Allergy-Immunology Clinics at Northwestern Medicine. Computed Tomography (CT) scans were graded according to defined methods (ref 35; incorporated by reference in its entirety) and history of taking PPIs listed in preoperative anesthesia records on the day of sinus surgery was obtained. Subject characteristics are included in Table 3. All subjects provided informed consent. The Institutional Review Board of Northwestern University-Feinberg School of Medicine approved this study. Tissue specimens including uncinate tissue (UT) and nasal polyp (NP), nasal lavage fluid, and epithelial scrapings from inferior turbinate (IT) and NP were obtained from subjects and prepared (refs. 36, 37; incorporated by reference in their entireties).

TABLE 3
Clinical characteristics of subjects

| | Control | | CRSsNP | | CRSwNP |
|---|---|---|---|---|---|
| Total no. of subjects (M/F) | 13 | (7/6) | 18 | (9/9) | 72 (47/25) |
| Age (y), median (range) | 41 | (19-78) | 40 | (20-69) | 43 (19-72) |

| | | | |
|---|---|---|---|
| Atopy (Y/N/U) | 0/12/1 | 10/4/4 | 46/16/10 |
| Asthma (Y/N/U) | 1/12/0 | 8/10/0 | 37/34/1 |
| Prior nasal surgery (Y/N) | 0/13 | 1/17 | 25/47 |

Methodology used:
Tissue extract

| | | | | | | | | |
|---|---|---|---|---|---|---|---|---|
| Tissue type, n (M/F) | UT, 7 | (3/4) | UT, 18 | (9/9) | UT, 39 | (26/13) | NP, 47 | (34/13) |
| Age (y), median (range) | 44 | (27-78) | 42 | (20-69) | 45 | (22-71) | 43 | (19-72) |

Nasal lavage fluid

| | | | | | |
|---|---|---|---|---|---|
| n (M/F) | 7 | (3/4) | 18 | (9/9) | 56 (38/18) |
| Age (y), median (range) | 44 | (27-78) | 42 | (20-69) | 45 (19-72) |

Cultured HNECs

| | | | | | | | |
|---|---|---|---|---|---|---|---|
| Origin, n (M/F) | IT, 6 | (4/2) | — | IT, 10 | (5/5) | Polyp, 6 | (4/2) |
| Age (y), median (range) | 34 | (19-59) | — | 46 | (32-63) | 38 | (35-40) |

### Measurement of Cytokines, Eotaxins, and ECP in Specimens

IL-4, IL-13, eotaxin-1, eotaxin-2, and eotaxin-3 levels were measured using the Milliplex Map kit (EMD Millipore, Billerica, Mass.) with a Luminex 200 instrument (Life Technologies, Gaithersburg, Md.). Eosinophil cationic protein (ECP) levels were measured using the Mesacup ECP Test (MBL International, Woburn, Mass.). Tissue concentrations of these mediators were normalized to the total protein concentration measured by the Bicinchoninic acid Protein Assay (Thermo Fisher Scientific, Watham, Mass.).

### Cell Culture

BEAS-2B, a human bronchial epithelial cell line transformed with a hybrid adenovirus 12-simian virus 40 was obtained from ATCC (CRL-9609, Manassas, Va.). Primary HNECs were collected by epithelial scraping of IT and NP and cultured. For cytokine (Peprotech, Rocky Hill, N.J.) stimulation, submerged cultured cells were treated with 1-100 ng/ml IL-13, 10 ng/ml IFN-γ, 100 ng/ml TNF or 50 ng/ml IL-17 for 6 h or 48 h. To study the effects of PPIs (Sigma-Aldrich, St Louis, Mo.) on cytokine-induced chemokines, cells were pretreated for 2 h with acid-activated omeprazole (0.1-50 μM) or other PPIs: lansoprazole, rabeprazole, pantoprazole, and esomeprazole (1-50 μM) prior to stimulation with 5 ng/ml IL-13. Additionally, SCH-28080 (1-50 μM; Sigma-Aldrich) was used with the same protocol. In experiments altering extracellular K⁺ concentration ([K⁺]ₑ), modified Ringer's solution that contained different contents of K⁺ (0-11.2 mM KCl, Table 4) was used as culture media. For mRNA stability assessment, actinomycin D (3 μg/ml, Sigma-Aldrich) was used and eotaxin-3 mRNA was measured using real-time PCR. Supernatants, whole cell lysates, and total RNAs were harvested for further analysis.

TABLE 4
Composition of solutions

| | Solution 1 | | | Solution 4 | | |
|---|---|---|---|---|---|---|
| Component | (K⁺-free RS) | Solution 2 | Solution 3 | (RS) | Solution 5 | Solution 6 |
| KCl (mM) | 0 | 2.82 | 4.23 | 5.63 | 8.45 | 11.26 |
| NaCl (mM) | 116.86 | 114.04 | 112.63 | 111.23 | 108.41 | 105.6 |
| CaCl₂ (mM) | 2.25 | 2.25 | 2.25 | 2.25 | 2.25 | 2.25 |
| NaHCO₃ (mM) | 2.38 | 2.38 | 2.38 | 2.38 | 2.38 | 2.38 |
| pH | 7.4 | 7.4 | 7.4 | 7.4 | 7.4 | 7.4 |

### ELISA

Eotaxin-1, eotaxin-2, and eotaxin-3 protein concentrations in supernatants were determined with the appropriate ELISA kits.

### Real-Time PCR and Western Blot

mRNA levels of eotaxin-1, eotaxin-2, eotaxin-3, CXCL1, CXCL10, ATP12A, and ATP4A in total RNAs isolated from cells were measured using quantitative real-time PCR. Western blots were performed to assess total signal transducer and activator of transcription 6 (STATE), phosphorylated-STAT6 (pSTAT6) and ATP12A protein in whole cell lysates (ref. 38; incorporated by reference in its entirety).

Intracellular pH (pHᵢ) Imaging

The pH-sensitive dye, pHrodo® Green AM intracellular pH indicator (Life Technologies) that increases its fluorescence with decreasing pHᵢ was used (ref 39; incorporated by reference in its entirety). Cells cultured in glass bottom microwell dishes (MatTek, Ashland, Mass.) were pre-treated with omeprazole or vehicle prior to 6 h IL-13 stimulation. Then cells were incubated with dye (5 μM) with live cell imaging solution (Life Technologies) at 37° C. for 30 minutes per manufacturer's instructions. Spinning disk confocal microscopy for live cells imaging was performed with Andor XDi Revolution (Andor Technologies, Belfast, UK). Fluorescence intensity was measured in 150 cells using Image J software (National Institutes of Health, Bethesda, Md.). For kinetic experiments, fluorescence intensity of cells cultured in 96-well plates with omeprazole, SCH-28080 or matched vehicle was measured at various times before and after IL-13 stimulation up to 1 h using the SpectraMax® Gemini EM Microplate Spectrofluorometer (Molecular devices, Sunnyvale, Calif.) at 485/538 nm (excitation/emission).

Small Interfering RNA (siRNA) Transfection

At 30-50% confluence, HNECs were transfected with 25 pmol ON-TARGETplus ATP12A siRNA or non-targeting negative control siRNA (Dharmacon™; GE Healthcare Life Sciences) in Lipofectamine RNAiMAX reagent (Life Technologies) per manufacturer's instructions. At 96 h post-transfection, cells were treated with omeprazole or vehicle, followed by IL-13 stimulation for 6 h. Knockdown efficiency was confirmed by using real-time PCR and Western blots.

### Example 1

### Levels of Type-2 Inflammatory Mediators and their Relationship with Tissue Eosinophilia and Radiographic Severity

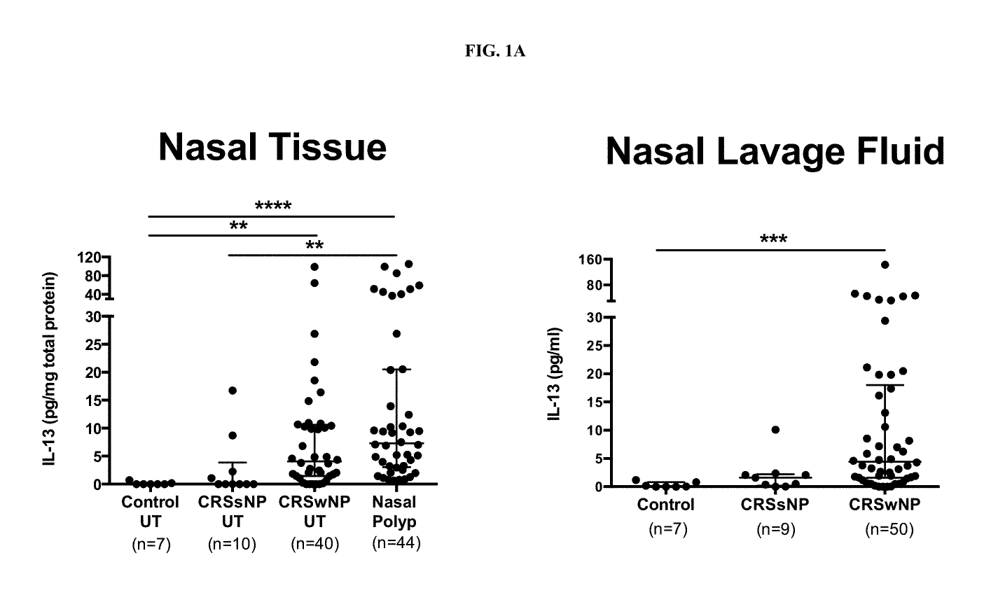

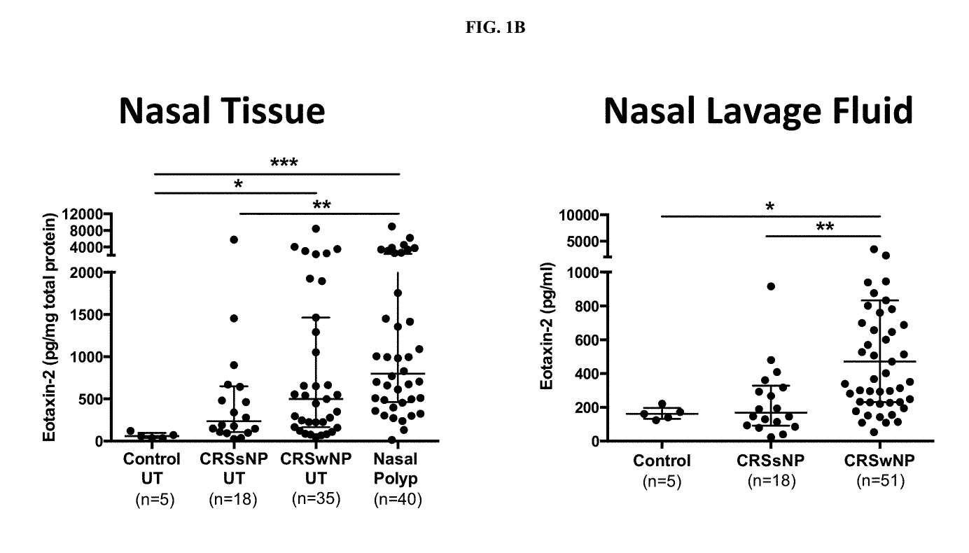

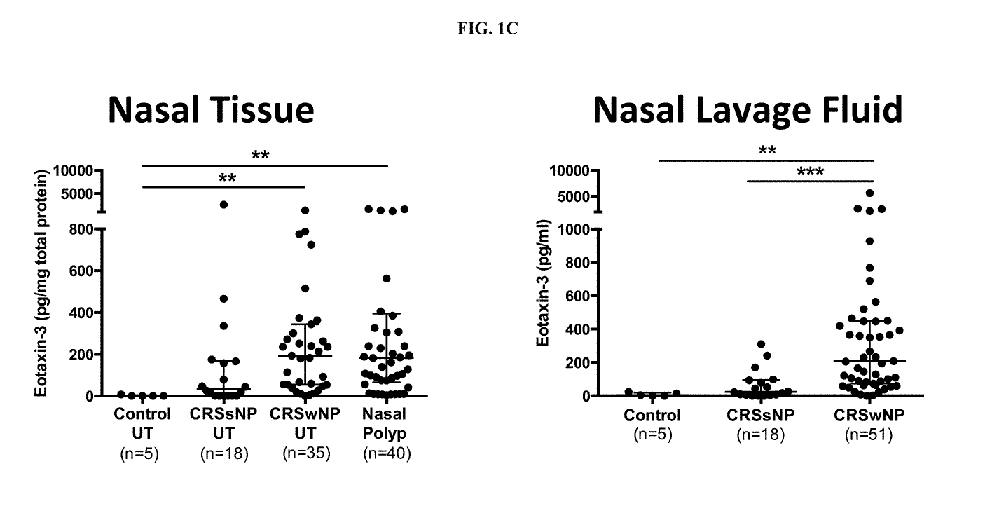

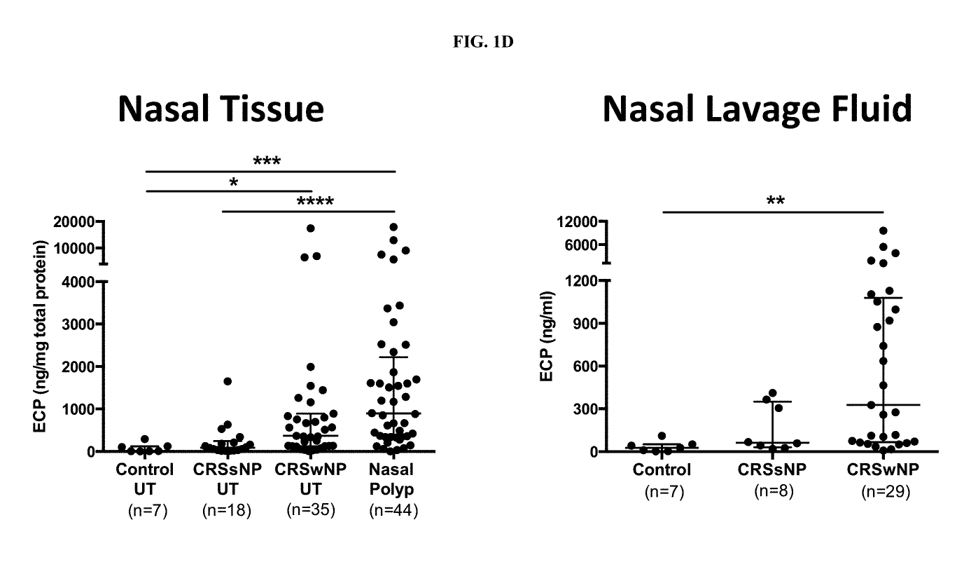

Experiments were conducted during development of embodiments herein to assess whether type-2 mediators in vivo levels were increased in patients with CRSwNP. IL-13 levels, but not IL-4 levels, were significantly elevated in CRSwNP UT and NP compared with control UT, with similar profiles were observed in nasal lavage fluid (FIG. 1, A). Among the eotaxins, eotaxin-2 (FIG. 1, B) and eotaxin-3 (FIG. 1, C) were significantly increased in tissues (UT and NP) and lavage fluid of CRSwNP compared with those of control. Eotaxin-1 levels were significantly elevated in NP only compared with control UT (median 61.0 versus 12.9 pg/mg total protein, respectively). ECP levels were significantly elevated in nasal tissues and secretions of CRSwNP compared with control (FIG. 1, D).

The correlations between tissue eosinophilia, as determined by ECP, and levels of type-2 mediators were then evaluated. ECP levels were significantly correlated with eotaxin-2, eotaxin-3, and IL-13 levels in UT and in lavage fluid among all subjects (Table 1). Next, radiographic severity (ref 35; incorporated by reference in its entirety) was correlated with these mediators in CRSwNP patients, and found that all eotaxins, IL-13 and ECP levels in UT were significantly correlated with CT scores (Table 1). Tissue and lavage eotaxin-2 and eotaxin-3 levels were also moderately correlated with UT IL-13 levels (Table 2). However, correlations carried out on type-2 mediators measured in NP were uncorrelated with local eosinophilia and radiographic severity (Table 5).

### Example 2

### Eotaxin-3 was the Dominant Eotaxin Induced by IL-13 in Airway Epithelial Cells

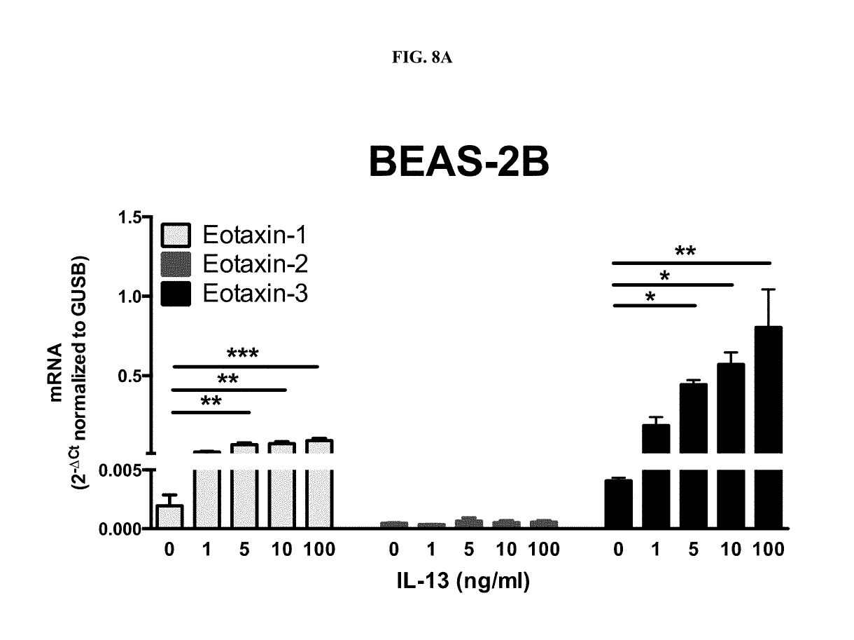

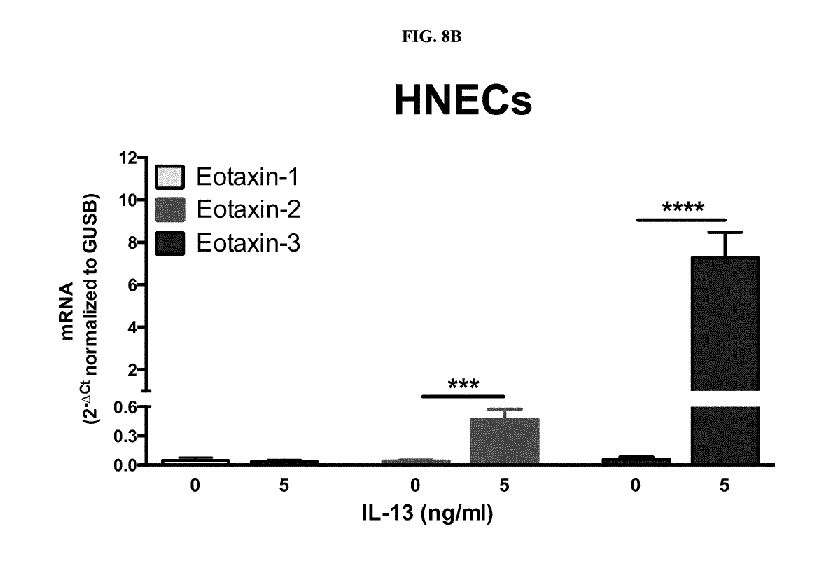

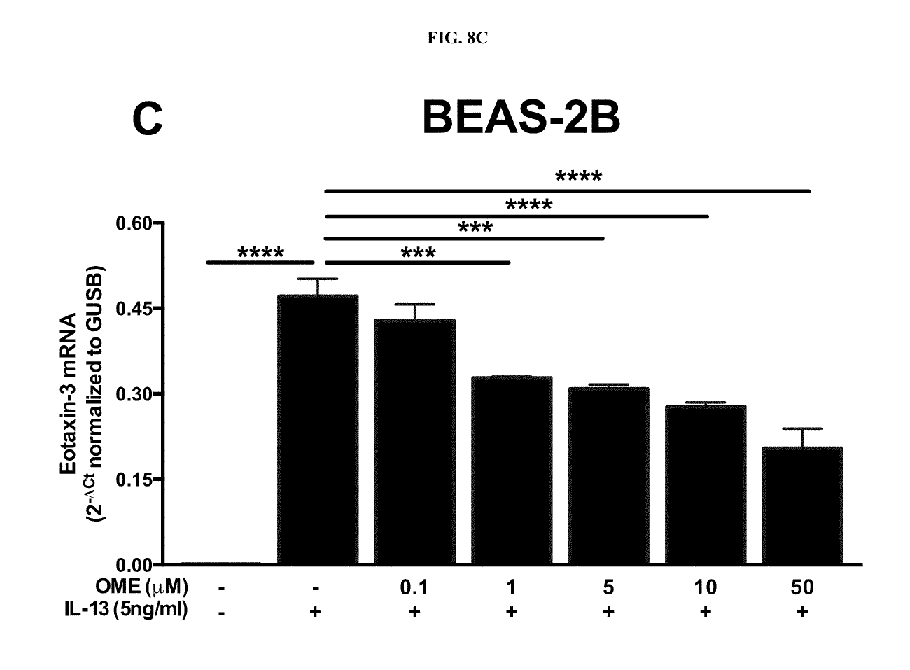

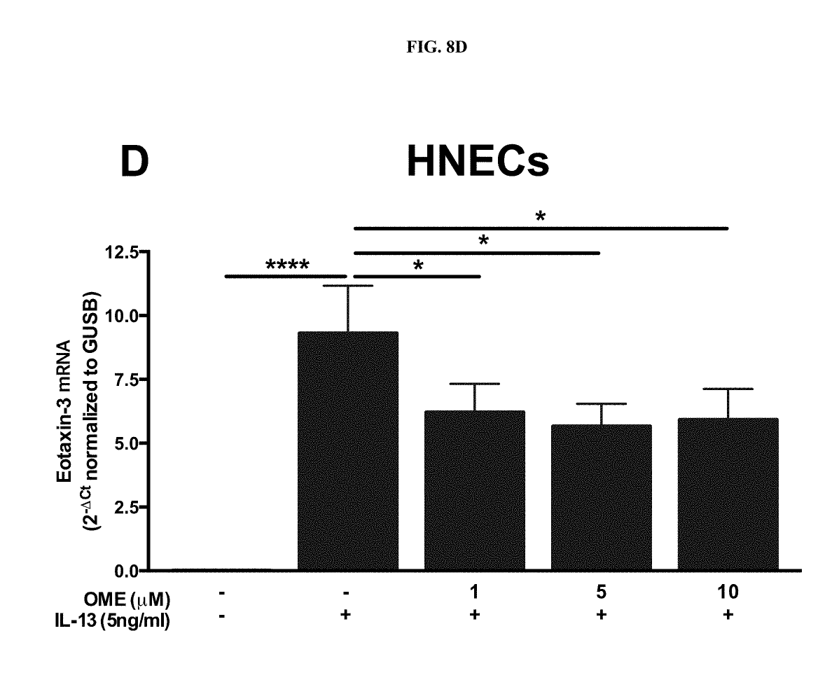

The effect of IL-13 on production of the eotaxins was evaluated in airway epithelial cells including HNECs and BEAS-2Bs in vitro. It was found that IL-13 significantly increased protein levels of all eotaxins in BEAS-2Bs (FIG. 2, A) and HNECs (FIG. 2, B). Notably, eotaxin-3 protein (FIGS. 2, A and B) and mRNA (FIGS. 8, A and B) expression were profoundly and concentration-dependently induced by IL-13 in both cell types. Considering that eotaxin-3 was most profoundly induced in vitro, and was highly expressed and positively correlated with surrogate markers of tissue eosinophilia in vivo, further experiments focuses on using eotaxin-3 as a target mediator for stimulation with IL-13.

### Example 3

### Omeprazole Inhibited IL-13-Induced Eotaxin-3 Production in Airway Epithelial Cells

Experiments were conducted during development of embodiments herein to determine whether omeprazole inhibits IL-13-induced eotaxin-3 in airway epithelial cells. It was found that IL-13-induced eotaxin-3 protein secretion was significantly inhibited in BEAS-2Bs and HNECs treated with omeprazole at concentrations as low as 5 μM and 1 μM, respectively (FIGS. 2, C and D). A similar pattern was observed in mRNA expression (FIGS. 8, C and D).

To ensure that the observed effect was specific to IL-13-induced eotaxin-3 and not a result of general inhibition of gene expression, mRNA expression of other chemokines (CXCL10, eotaxin-1, and CXCL1) was measured in response to IFN-γ, TNF-α, and IL-17, respectively, with or without omeprazole pre-treatment. These chemokines were significantly induced by their respective cytokines (refs. 16, 40-41; incorporated by reference in their entireties) but their expression was not inhibited by omeprazole or other tested PPIs in BEAS-2Bs (FIG. 9).

### Example 4

### Association of PPI Use and In Vivo Eotaxins Levels in CRS Patients

Since the inhibitory effect of omeprazole on IL-13-induced eotaxin-3 expression in airway epithelial cells, experiments were conducted to determine if in vitro findings have corresponding in vivo effects. Upon medical record review, nine (17%) of the CRS patients were identified as taking PPIs including omeprazole (n=5), esomeprazole (n=1), lansoprazole (n=2), and rabeprazole (n=1) at the time of sinus surgery. Subjects taking PPIs had significantly lower eotaxin-2 and eotaxin-3 levels in UT compared with subjects without PPIs (FIG. 3). Similar trends were observed in tissue eotaxin-1 and ECP level.

### Example 5

### Other PPIs and SCH-28080 Inhibited IL-13-Induced Eotaxin-3 Expression

Like omeprazole, other PPIs, including lansoprazole, rabeprazole, pantoprazole, and esomeprazole, showed dose-dependent inhibitory effects on IL-13-induced eotaxin-3 protein secretion, indicating a class effect of PPIs (FIG. 4, A). Moreover, when the extrapolated relative potencies of PPIs for inhibiting IL-13-induced eotaxin-3, were compared with their published potencies as inhibitors of gastric acid secretion (ref 42; incorporated by reference in its entirety), there was a strong positive correlation between these two different effects (r=0.91, P=0.03; FIG. 4, B). It was found that SCH-28080 also significantly inhibited IL-13-induced eotaxin-3 levels (FIG. 4, C). SCH-28080 is mechanistically unrelated to PPIs in that it inhibits H,K-ATPases via competitive interactions with K⁺ (refs. 43-44; incorporated by reference in their entireties), while PPIs function via binding to sulfhydryl groups of the H,K-ATPase (ref. 43; incorporated by reference in its entirety). Given these findings, it is contemplated that H,K-ATPase activity regulates IL-13-induced eotaxin-3 expression.

### Example 6

### Non-Gastric H,K-ATPase: Implication for IL-13-Induced Responses and Effect of PPIs

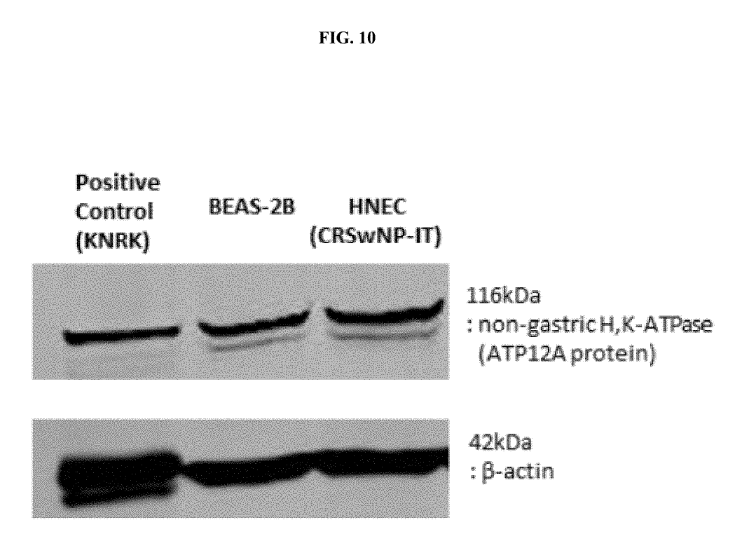

In humans, P-type ATPases comprise numerous ion-pumps but only two H,K-ATPases have been described. The gastric H,K-ATPase (gH,K-ATPase, encoded by the ATP4A gene), is the classic target of PPIs in the stomach but was not expressed by airway epithelial cells. In contrast, the non-gastric H,K-ATPase (ngH,K-ATPase, encoded by the ATP12A gene) has been found in kidney, prostate, lung and nasal epithelium, and represented a possible candidate (refs. 45-47; incorporated by reference in their entireties). The presence of the catalytic a-subunit of ngH,K-ATPase was confirmed in BEAS-2Bs and HNECs (FIG. 10). The ngH,K-ATPase exchanges extracellular K⁺ for intracellular H⁺ (ref 44; incorporated by reference in its entirety). To test whether activated ngH,K-ATPase induces intracellular alkalinization, pHᵢ was measured and it was found that IL-13-stimulated cells showed significantly decreased fluorescence compared with unstimulated cells, indicating IL-13-induced increased intracellular pH (FIG. 5, A). Moreover, omeprazole significantly attenuated this effect compared with vehicle (FIG. 5, A). In kinetic studies, intracellular alkalinization became apparent as early as 20 minutes after IL-13 stimulation and was blunted in omeprazole- or SCH-28080-treated cells (FIGS. 5, B and 11, respectively).

It was contemplated that IL-13-mediated responses depend on [K⁺]ₑ to facilitate ngH,K-ATPase activity. As demonstrated in FIG. 5C, IL-13-mediated eotaxin-3 mRNA induction was influenced by [K⁺]ₑ and was completely eliminated in [K⁺]ₑ-free conditions, further demonstrating the role of ngH,K-ATPase in mediating IL-13-induced gene expression.

### Example 7

### Knockdown of ATP12A

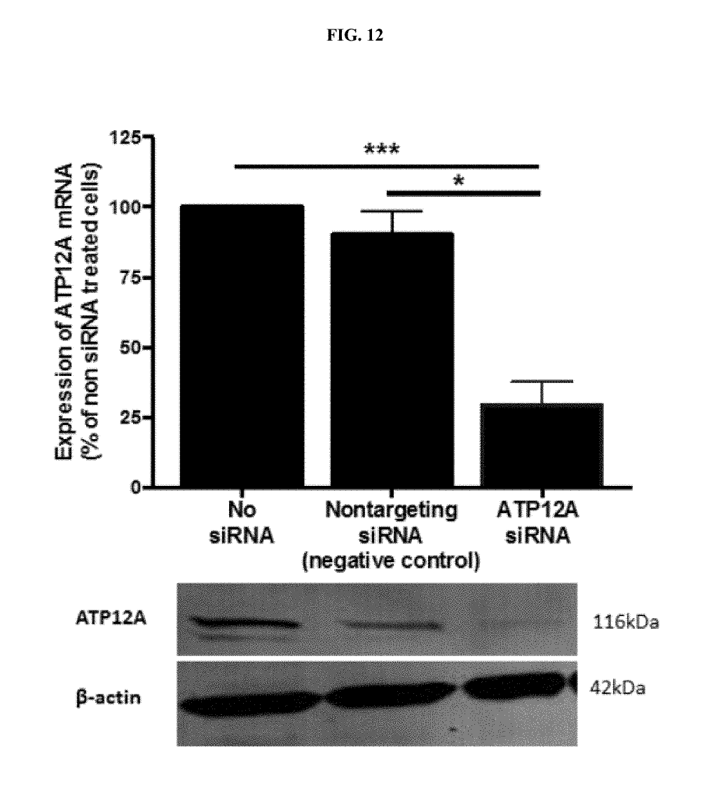

The expression of ATP12A was directly disrupted using a siRNA knockdown approach. Overall knockdown efficiency for ATP12A mRNA was 71% in HNECs (FIG. 12). Induction of eotaxin-3 by IL-13 was significantly reduced in ATP12A siRNA-transfected cells compared with non-targeting siRNA-transfected cells, but an additive effect of omeprazole was not observed in ATP12A siRNA-transfected cells (FIG. 5, D).

### Example 8

### Effect of Omeprazole on STAT6 Phosphorylation and Eotaxin-3 mRNA Stability

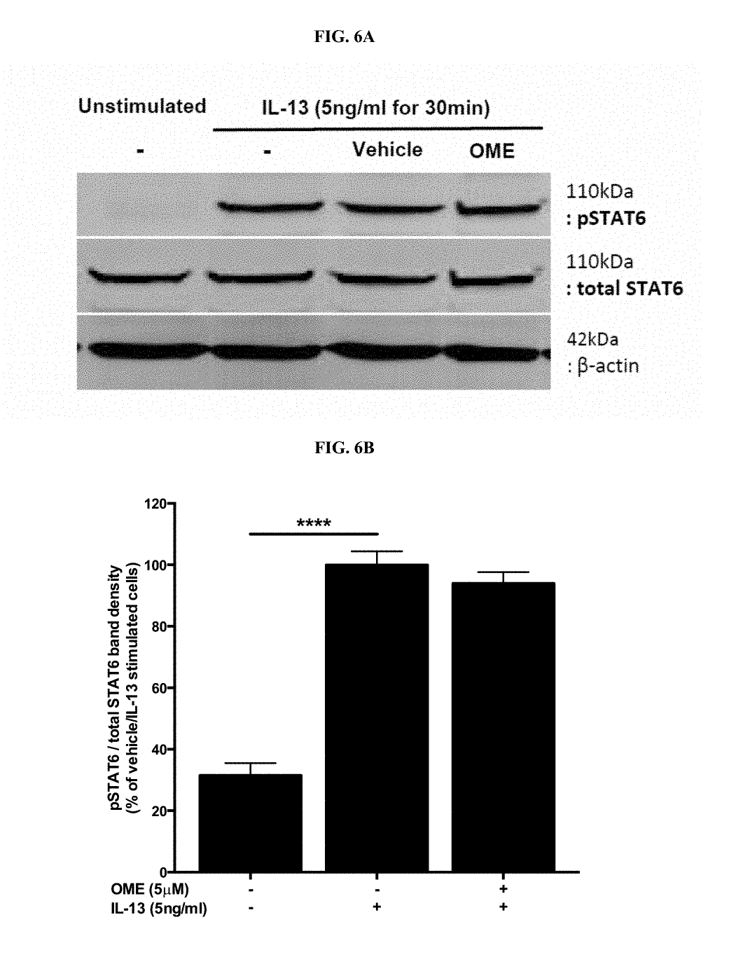

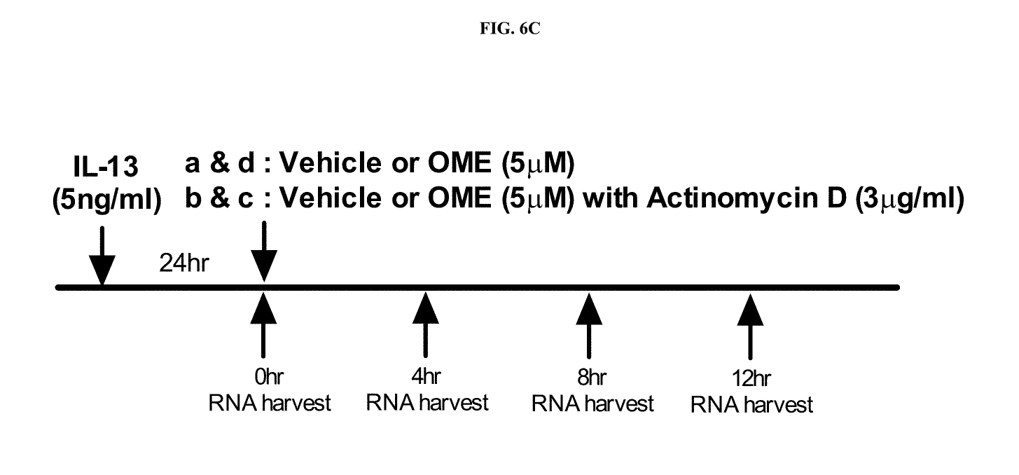

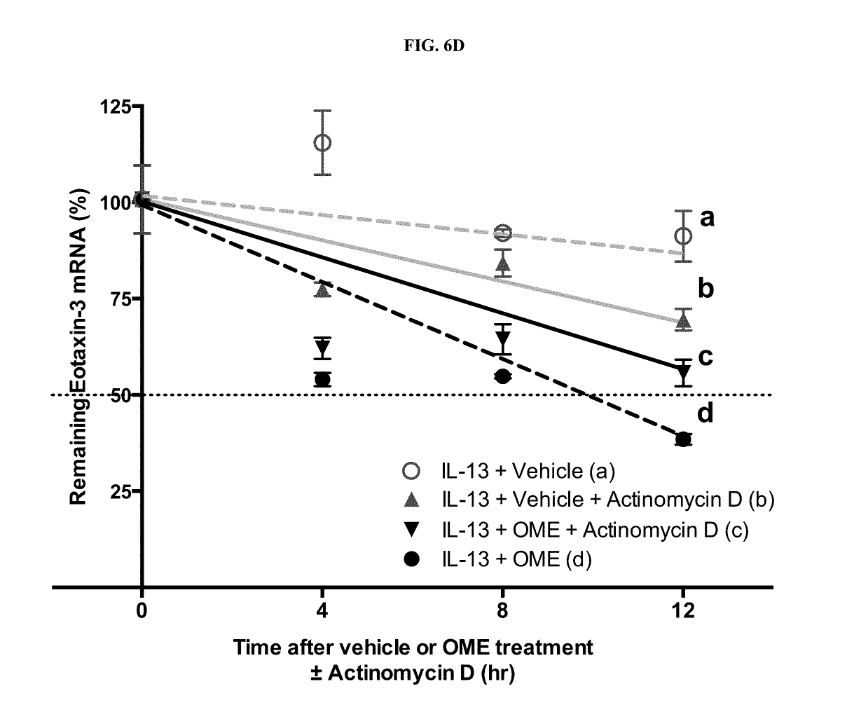

The effect of omeprazole on STAT6 phosphorylation was evaluated. IL-13-induced pSTAT6 was not significantly inhibited by omeprazole (FIGS. 6, A and B).

It was next assessed whether omeprazole influenced IL-13-induced eotaxin-3 mRNA stability by utilizing actinomycin D, which inhibits de novo transcription (FIG. 6, C) (ref 48; incorporated by reference in its entirety). IL-13-induced eotaxin-3 mRNA expression was relatively stable without omeprazole or actinomycin D (FIG. 6, D, line a). Omeprazole significantly accelerated decline of eotaxin-3 mRNA levels over the following 12 h (FIG. 6, D, lines a vs. d, at 12 h). In the presence of actinomycin D, omeprazole had a lesser effect but still enhanced eotaxin-3 mRNA decay compared to vehicle (FIG. 6, D, lines c vs. b,), indicating post-transcriptional regulation by omeprazole. However, when comparing the effect of omeprazole with or without actinomycin D, a lesser magnitude of eotaxin-3 mRNA decay was observed in the presence of actinomycin D (FIG. 6, D, line c) compared to that of omeprazole alone (FIG. 6, D, line d, after 8 h), indicating that inhibition of eotaxin-3 mRNA by omeprazole might in part be related to decreased de novo transcription as well as increased post-transcriptional degradation.

### Example 9

### Complete Genetic Inhibition of ATP12A by CRISPR-CAS9

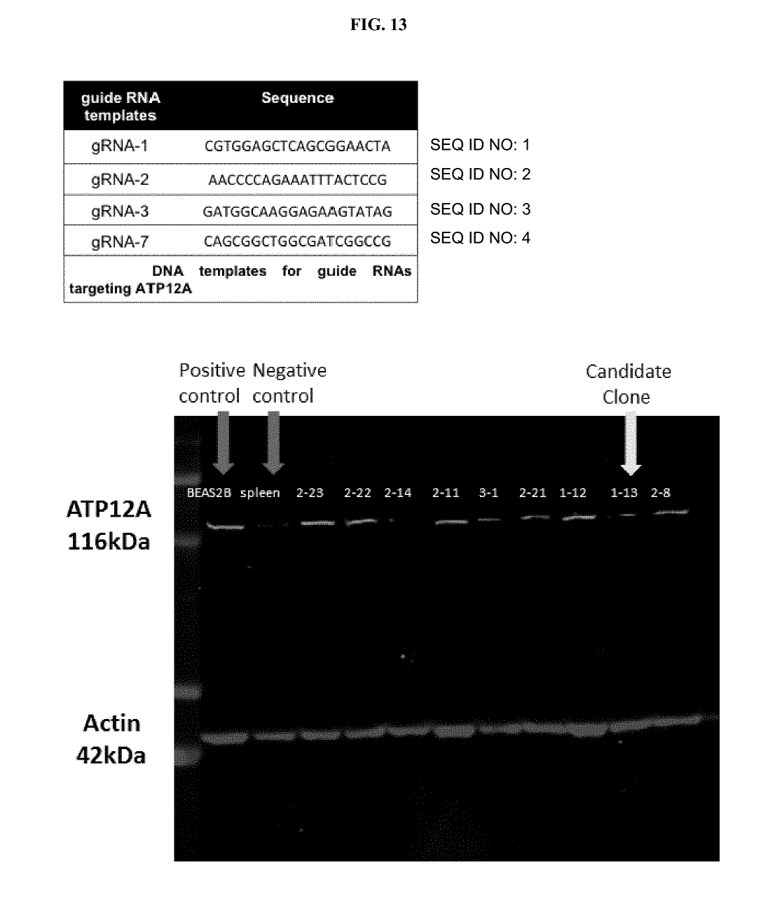

ATP12A was knocked out in the BEAS2B cell line using a lentivirus with targeting guide RNA-2 using the CRISPR-Cas9 technique (FIG. 13). Synthetic gRNAs templates (CRISPR crRNA, from IDT)) were delivered using the transient tranfection reagent TransIT-X2 (Minis Bio, Madison, Wis.) into BEAS2B cells stably expressing the CAS9 protein (S. pyogenes CRISPR-Cas9). A clone was identified that completely knocked out ATP12A using Western blot (FIG. 13). BEAS2B ATP12A wild-type cells and ATP12A knockout cells were stimulated with 5 ng/ml of IL-13 and eotaxin-3 protein secretion was compared.

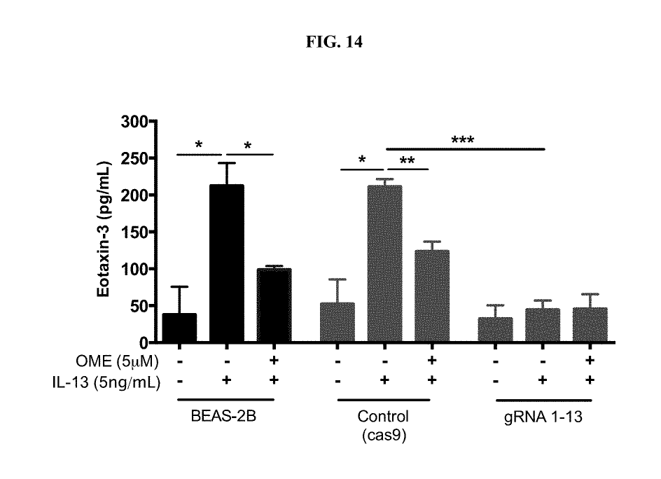

Compared to ATP12A wild type BEAS2B cells and cas9-expressing BEAS2B cells without guide RNA transfection, the ATP12A knockout BEAS2B cells showed no increase in eotaxin-3 expression following stimulation (FIG. 14). 1. These experiments demonstrate that complete inhibition of ATP12A using a genetic manipulation technique (CRISPR-CAS9) of a human epithelial cell line (BEAS-2B) eliminated IL-13-induced eotaxin-3 gene expression in vitro.

### Example 10

### Suppression of ATP12A Function by Pharmacologic Inhibition

Submerged cultured primary nasal epithelial cells (NECs) were treated with 5 ng/ml of IL-13 with or without pretreatment for 2 h with acid-activated omeprazole (5 μM). After 6 hours of stimulation, the cells were harvested and mRNA levels of several candidate genes including periostin (POSTN), an extracellular matrix protein were determined by qRT-PCR, while the expression of several other genes including arachidonate 15-lipoxygenase (ALOX15) an enzyme involved in arachidonic acid metabolism and claudin-5 (CLDN5) an epithelial tight junction protein were determined by RNA-Seq. Separately, NECs were grown in transwells at air-liquid interface to induce differentiation of epithelium into cilitated differentiated epithelium. 5 ng/ml of basal IL-13 was then applied and gene expression of mucin 5AC (MUC5AC) was measured at 24 hrs after stimulation with or without pretreatment for 2 h with acid-activated omeprazole (5 μM).

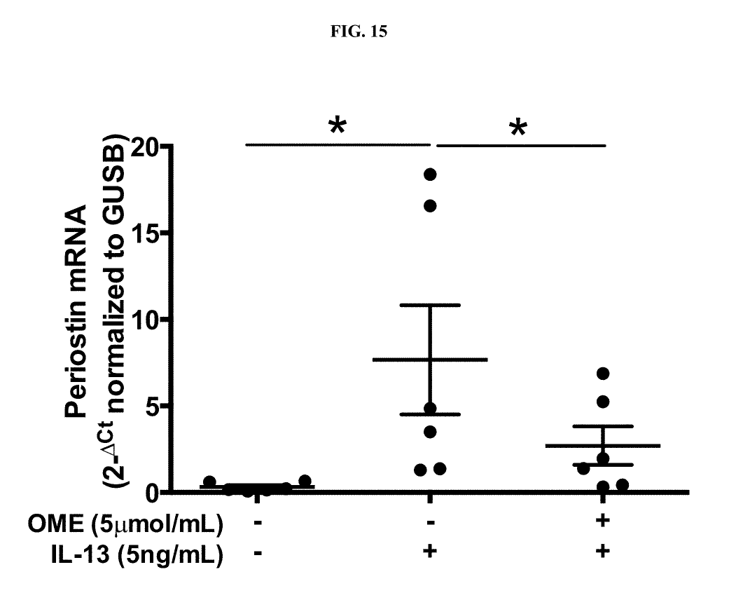

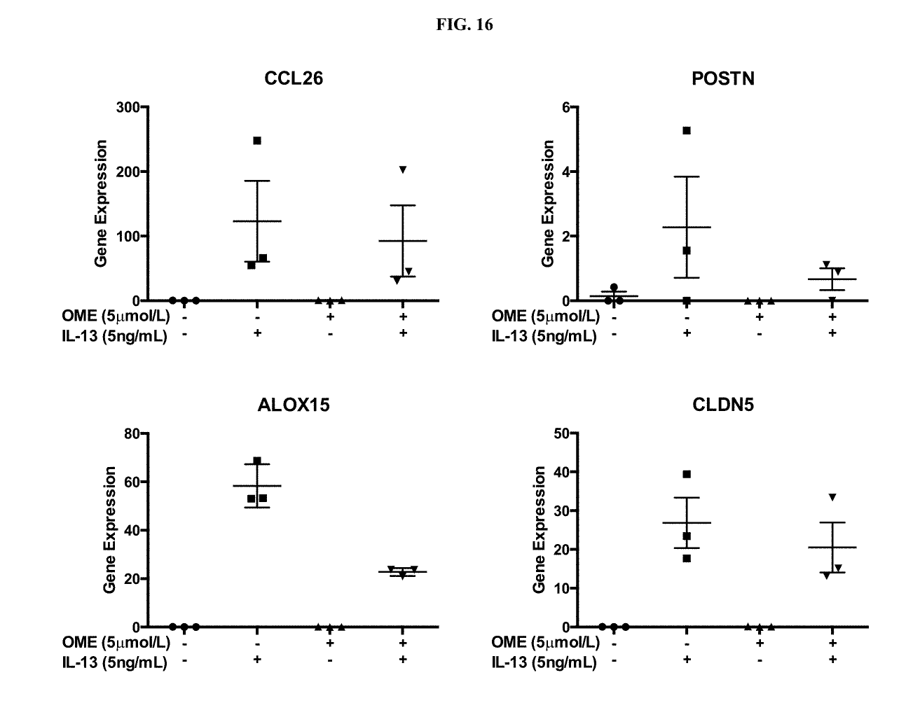

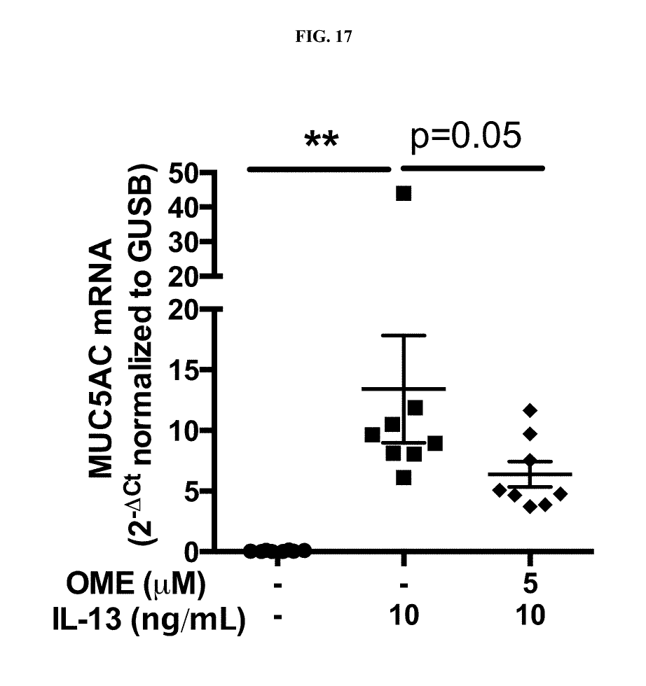

In addition to suppressing IL-13-induced eotaxin-3 production, the suppression of non-gastric H+/K+ATPase by omeprazole demonstrated a significant reduction in the pathogenic effects of IL-13. Suppression of non-gastric H+/K+ATPase also reduces IL-13 induced barrier disruption, arachidonic acid metabolism and mucus hyper-secretion (FIGS. 15-17). 1. These experiments demonstrate that suppression of ATP12A function using pharmacologic inhibition suppresses other known pathogenic effects of IL-13 on epithelial cells including airway epithelial remodeling, leukotriene metabolism and mucus production.

### Example 11

### Type-2 Cytokines Drive an Acidification of Airway Surface Liquid that is Reversed by Inhibition of the Non-Gastric H+/K+ATPase

NECs were grown at air-liquid interface to induce differentiation of epithelium into cilitated differentiated epithelium. When confluent, the cell culture was replaced with unbuffered live cell imaging solution (LCIS). 5 ng/ml of basally applied IL-13 was then applied with or without pretreatment for 1 hr with acid-activated omeprazole (5 μM). pH measurements were made by applying 50 μl of a SNARF-dextran ratiometric pH dye and read on a spectrofluorometer. Separately, the pH of nasal secretions from the middle meatus were measured using a micro pH meter. Type-2 cytokines were then measured using a cytometric bead array (Millipore).

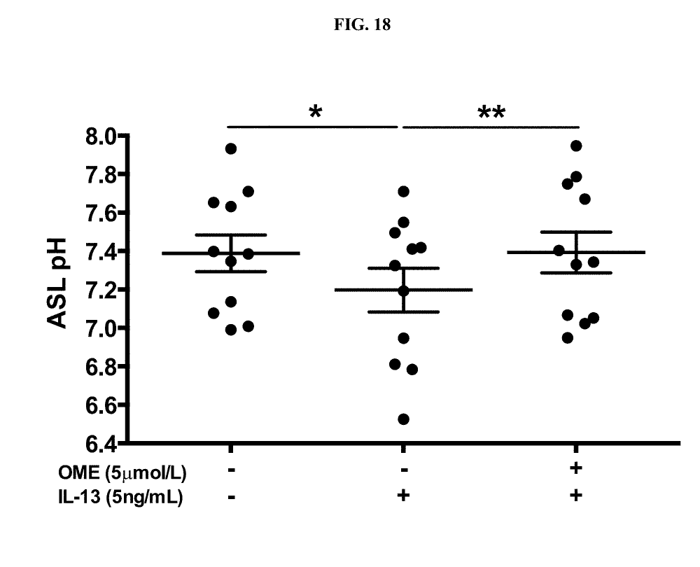

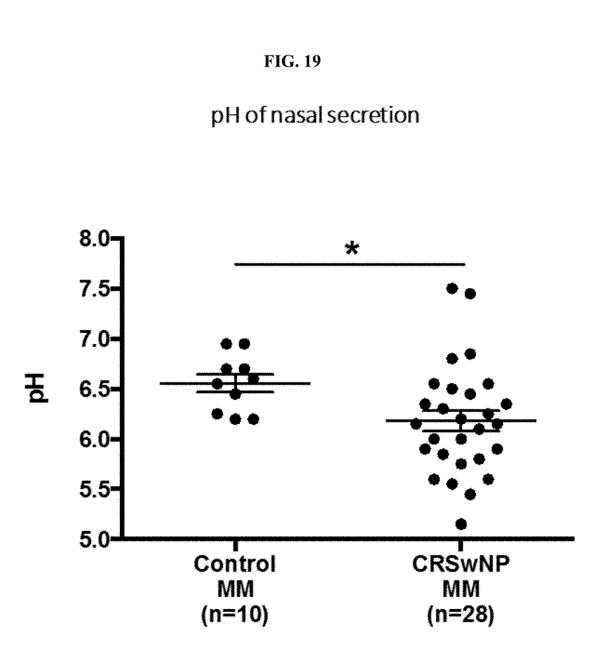

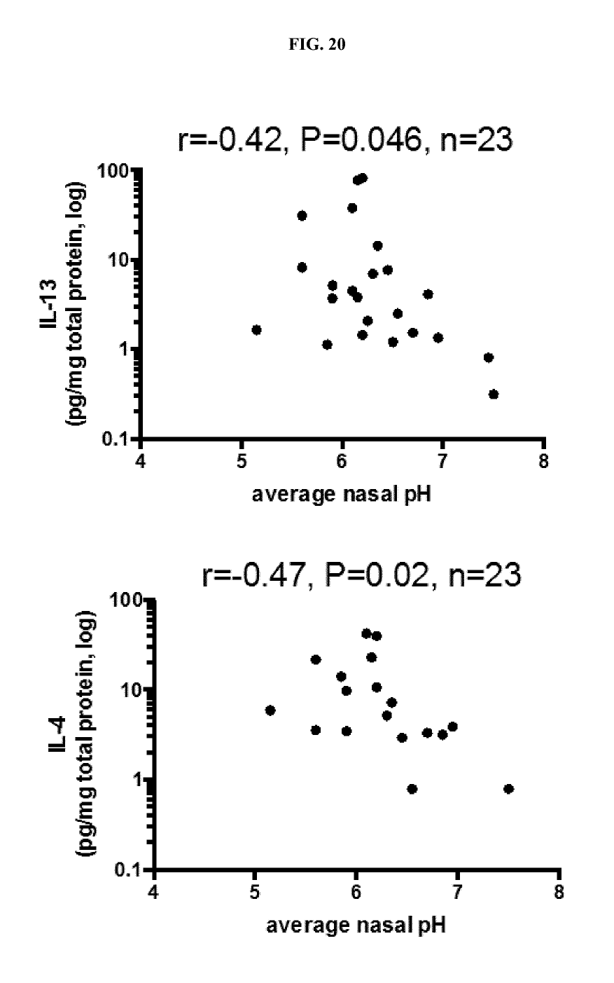

Following IL-13 exposure, the pH of the airway surface liquid over NEC cells was significantly acidified compared to that over IL-13 untreated cells (FIG. 18). Omeprazole prevented the acidification of airway surface liquid. The pH of airway secretions in CRSwNP patients was significantly lower than the airway secretions from control patients (FIG. 19). Airway pH was significantly negatively correlated with type-2 cytokines like IL-13 and IL-4 (FIG. 20). These experiments demonstrate that type-2 cytokines drive an acidification of airway surface liquid that is reversed by inhibition of the non-gastric H+/K+ATPase, as evidenced by the in vitro and in vivo studies herein.

All publications and patents mentioned herein are incorporated by reference in their entireties. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

The following references, some of which are cited above by number, are herein incorporated by reference in their entireties.
1. Dykewicz M S, Hamilos D L. Rhinitis and sinusitis. J Allergy Clin Immunol 2010; 125:S103-15.
2. Meltzer E O, Hamilos D L, Hadley J A, Lanza D C, Marple B F, Nicklas R A, et al. Rhinosinusitis: establishing definitions for clinical research and patient care. J Allergy Clin Immunol 2004; 114:155-212.
3. Kern R C, Conley D B, Walsh W, Chandra R, Kato A, Tripathi-Peters A, et al. Perspectives on the etiology of chronic rhinosinusitis: an immune barrier hypothesis. Am J Rhinol 2008; 22:549-59.
4. Van Crombruggen K, Zhang N, Gevaert P, Tomassen P, Bachert C. Pathogenesis of chronic rhinosinusitis: inflammation. J Allergy Clin Immunol 2011; 128:728-32.
5. Tokunaga T, Sakashita M, Haruna T, Asaka D, Takeno S, Ikeda H, et al. Novel scoring system and algorithm for classifying chronic rhinosinusitis: the JESREC Study. Allergy 2015.
6. Nakayama T, Yoshikawa M, Asaka D, Okushi T, Matsuwaki Y, Otori N, et al. Mucosal eosinophilia and recurrence of nasal polyps—new classification of chronic rhinosinusitis. Rhinology 2011; 49:392-6.
7. Soler Z M, Sauer D, Mace J, Smith T L. Impact of mucosal eosinophilia and nasal polyposis on quality-of-life outcomes after sinus surgery. Otolaryngol Head Neck Surg 2010; 142:64-71.
8. Larose M C, Chakir J, Archambault A S, Joubert P, Provost V, Laviolette M, et al. Correlation between CCL26 production by human bronchial epithelial cells and airway eosinophils: Involvement in patients with severe eosinophilic asthma. J Allergy Clin Immunol 2015.
9. Rosenberg H F, Phipps S, Foster P S. Eosinophil trafficking in allergy and asthma. J Allergy Clin Immunol 2007; 119:1303-10; quiz 11-2.
10. Lamkhioued B, Garcia-Zepeda E A, Abi-Younes S, Nakamura H, Jedrzkiewicz S, Wagner L, et al. Monocyte chemoattractant protein (MCP)-4 expression in the airways of patients with asthma. Induction in epithelial cells and mononuclear cells by proinflammatory cytokines. Am J Respir Crit Care Med 2000; 162:723-32.
11. Schleimer R P, Kato A, Peters A, Conley D, Kim J, Liu M C, et al. Epithelium, inflammation, and immunity in the upper airways of humans: studies in chronic rhinosinusitis. Proc Am Thorac Soc 2009; 6:288-94.
12. Laoukili J, Perret E, Willems T, Minty A, Parthoens E, Houcine O, et al. IL-13 alters mucociliary differentiation and ciliary beating of human respiratory epithelial cells. J Clin Invest 2001; 108:1817-24.
13. Kuperman D A, Huang X, Koth L L, Chang G H, Dolganov G M, Zhu Z, et al. Direct effects of interleukin-13 on epithelial cells cause airway hyperreactivity and mucus overproduction in asthma. Nat Med 2002; 8:885-9.
14. Pope S M, Brandt E B, Mishra A, Hogan S P, Zimmermann N, Matthaei K I, et al. IL-13 induces eosinophil recruitment into the lung by an IL-5- and eotaxin-dependent mechanism. J Allergy Clin Immunol 2001; 108:594-601.
15. Kelly M, Hwang J M, Kubes P. Modulating leukocyte recruitment in inflammation. J Allergy Clin Immunol 2007; 120:3-10.
16. Komiya A, Nagase H, Yamada H, Sekiya T, Yamaguchi M, Sano Y, et al. Concerted expression of eotaxin-1, eotaxin-2, and eotaxin-3 in human bronchial epithelial cells. Cell Immunol 2003; 225:91-100.
17. Blanchard C, Durual S, Estienne M, Emami S, Vasseur S, Cuber J C. Eotaxin-3/CCL26 gene expression in intestinal epithelial cells is up-regulated by interleukin-4 and interleukin-13 via the signal transducer and activator of transcription 6. Int J Biochem Cell Biol 2005; 37:2559-73.
18. Heiman A S, Abonyo B O, Darling-Reed S F, Alexander M S. Cytokine-stimulated human lung alveolar epithelial cells release eotaxin-2 (CCL24) and eotaxin-3 (CCL26). J Interferon Cytokine Res 2005; 25:82-91.
19. Neilsen C V, Bryce P J. Interleukin-13 directly promotes oesophagus production of CCL11 and CCL24 and the migration of eosinophils. Clin Exp Allergy 2010; 40:427-34.
20. Provost V, Larose M C, Langlois A, Rola-Pleszczynski M, Flamand N, Laviolette M. CCL26/eotaxin-3 is more effective to induce the migration of eosinophils of asthmatics than CCL11/eotaxin-1 and CCL24/eotaxin-2. J Leukoc Biol 2013; 94:213-22.
21. Blanchard C, Wang N, Stringer K F, Mishra A, Fulkerson P C, Abonia J P, et al. Eotaxin-3 and a uniquely conserved gene-expression profile in eosinophilic esophagitis. J Clin Invest 2006; 116:536-47.
22. Shaw J L, Fakhri S, Citardi M J, Porter P C, Corry D B, Kheradmand F, et al. IL-33-responsive innate lymphoid cells are an important source of IL-13 in chronic rhinosinusitis with nasal polyps. Am J Respir Crit Care Med 2013; 188:432-9.
23. Mjosberg J M, Trifari S, Crellin N K, Peters C P, van Drunen C M, Piet B, et al. Human I L-25- and IL-33-responsive type 2 innate lymphoid cells are defined by expression of CRTH2 and CD161. Nat Immunol 2011; 12:1055-62.
24. Beck L A, Stellato C, Beall L D, Schall T J, Leopold D, Bickel C A, et al. Detection of the chemokine RANTES and endothelial adhesion molecules in nasal polyps. J Allergy Clin Immunol 1996; 98:766-80.
25. Stevens W W, Ocampo C J, Berdnikovs S, Sakashita M, Mandavinia M, Suh L, et al. Cytokines in Chronic Rhinosinusitis: Role in Eosinophilia and Aspirin Exacerbated Respiratory Disease. Am J Respir Crit Care Med 2015.
26. Gevaert P, Van Bruaene N, Cattaert T, Van Steen K, Van Zele T, Acke F, et al. Mepolizumab, a humanized anti-IL-5 mAb, as a treatment option for severe nasal polyposis. J Allergy Clin Immunol 2011; 128:989-95-8.
27. Bachert C, Mannent L, Naclerio R M, Mullol J, Ferguson B J, Gevaert P, et al. Effect of Subcutaneous Dupilumab on Nasal Polyp Burden in Patients With Chronic Sinusitis and Nasal Polyposis: A Randomized Clinical Trial. JAMA 2016; 315:469-79.
28. Bachert C, Zhang L, Gevaert P. Current and future treatment options for adult chronic rhinosinusitis: Focus on nasal polyposis. J Allergy Clin Immunol 2015; 136:1431-40.
29. Cheng E, Zhang X, Huo X, Yu C, Zhang Q, Wang D H, et al. Omeprazole blocks eotaxin-3 expression by oesophageal squamous cells from patients with eosinophilic oesophagitis and GORD. Gut 2013; 62:824-32.
30. Zhang X, Cheng E, Huo X, Yu C, Zhang Q, Pham T H, et al. Omeprazole blocks STATE binding to the eotaxin-3 promoter in eosinophilic esophagitis cells. PLoS One 2012; 7:e50037.
31. Park J Y, Zhang X, Nguyen N, Souza R F, Spechler S J, Cheng E. Proton pump inhibitors decrease eotaxin-3 expression in the proximal esophagus of children with esophageal eosinophilia. PLoS One 2014; 9:e101391.
32. Lucendo A J, Arias A, Molina-Infante J. Efficacy of Proton Pump Inhibitor Drugs for Inducing Clinical and Histologic Remission in Patients With Symptomatic Esophageal Eosinophilia: A Systematic Review and Meta-Analysis. Clin Gastroenterol Hepatol 2015.
33. Padia R, Curtin K, Peterson K, Orlandi R R, Alt J. Eosinophilic esophagitis strongly linked to chronic rhinosinusitis. Laryngoscope 2015.
34. Pearlman A N, Conley D B. Review of current guidelines related to the diagnosis and treatment of rhinosinusitis. Curr Opin Otolaryngol Head Neck Surg 2008; 16:226-30.
35. Okushi T, Nakayama T, Morimoto S, Arai C, Omura K, Asaka D, et al. A modified Lund-Mackay system for radiological evaluation of chronic rhinosinusitis. Auris Nasus Larynx 2013; 40:548-53.
36. Tan B K, Li Q Z, Suh L, Kato A, Conley D B, Chandra R K, et al. Evidence for intranasal antinuclear autoantibodies in patients with chronic rhinosinusitis with nasal polyps. J Allergy Clin Immunol 2011; 128:1198-206 el.
37. Kato A, Peters A, Suh L, Carter R, Harris K E, Chandra R, et al. Evidence of a role for B cell-activating factor of the TNF family in the pathogenesis of chronic rhinosinusitis with nasal polyps. J Allergy Clin Immunol 2008; 121:1385-92, 92 el-2.
38. Chustz R T, Nagarkar D R, Poposki J A, Favoreto S, Jr., Avila P C, Schleimer R P, et al. Regulation and function of the IL-1 family cytokine IL-1F9 in human bronchial epithelial cells. Am J Respir Cell Mol Biol 2011; 45:145-53.
39. Arppe R, Nareoja T, Nylund S, Mattsson L, Koho S, Rosenholm J M, et al. Photon upconversion sensitized nanoprobes for sensing and imaging of pH. Nanoscale 2014; 6:6837-43.
40. Sauty A, Dziejman M, Taha R A, Iarossi A S, Neote K, Garcia-Zepeda E A, et al. The T cell-specific CXC chemokines IP-10, Mig, and I-TAC are expressed by activated human bronchial epithelial cells. J Immunol 1999; 162:3549-58.
41. Huang F, Kao C Y, Wachi S, Thai P, Ryu J, Wu R. Requirement for both JAK-mediated PI3K signaling and ACT1/TRAF6/TAK1-dependent NF-kappaB activation by IL-17A in enhancing cytokine expression in human airway epithelial cells. J Immunol 2007; 179:6504-13.
42. Kirchheiner J, Glatt S, Fuhr U, Klotz U, Meineke I, Seufferlein T, et al. Relative potency of proton-pump inhibitors-comparison of effects on intragastric pH. Eur J Clin Pharmacol 2009; 65:19-31.
43. Shin J M, Munson K, Vagin O, Sachs G. The gastric H K-ATPase: structure, function, and inhibition. Pflugers Arch 2009; 457:609-22.
44. Modyanov N N, Mathews P M, Grishin A V, Beguin P, Beggah A T, Rossier B C, et al. Human ATP1AL1 gene encodes a ouabain-sensitive H-K-ATPase. Am J Physiol 1995; 269:C992-7.
45. Coakley R D, Grubb B R, Paradiso A M, Gatzy J T, Johnson L G, Kreda S M, et al. Abnormal surface liquid pH regulation by cultured cystic fibrosis bronchial epithelium. Proc Natl Acad Sci USA 2003; 100:16083-8.
46. Altman K W, Kinoshita Y, Tan M, Burstein D, Radosevich J A. Western blot confirmation of the H+/K+-ATPase proton pump in the human larynx and submandibular gland. Otolaryngol Head Neck Surg 2011; 145:783-8.
47. Pestov N B, Korneenko T V, Shakhparonov M I, Shull G E, Modyanov N N. Loss of acidification of anterior prostate fluids in Atp12a-null mutant mice indicates that nongastric H-K-ATPase functions as proton pump in vivo. Am J Physiol Cell Physiol 2006; 291:C366-74.
48. Heller N M, Matsukura S, Georas S N, Boothby M R, Rothman P B, Stellato C, et al. Interferon-gamma inhibits STATE signal transduction and gene expression in human airway epithelial cells. Am J Respir Cell Mol Biol 2004; 31:573-82.
49. Tajiri T, Matsumoto H, Hiraumi H, Ikeda H, Morita K, Izuhara K, et al. Efficacy of omalizumab in eosinophilic chronic rhinosinusitis patients with asthma. Ann Allergy Asthma Immunol 2013; 110:387-8.
50. Lam M, Hull L, McLachlan R, Snidvongs K, Chin D, Pratt E, et al. Clinical severity and epithelial endotypes in chronic rhinosinusitis. Int Forum Allergy Rhinol 2013; 3:121-8.
51. De Corso E, Baroni S, Romitelli F, Luca L, Di Nardo W, Passali G C, et al. Nasal lavage CCL24 levels correlate with eosinophils trafficking and symptoms in chronic sino-nasal eosinophilic inflammation. Rhinology 2011; 49:174-9.
52. Gu Z, Jin M, Cao Z. Role of eotaxin-3 in chronic rhinosinusitis with nasal polyps. Otolaryngol Head Neck Surg 2011; 145:324-6.
53. Takabayashi T, Kato A, Peters A T, Hulse K E, Suh L A, Carter R, et al. Increased expression of factor XIII-A in patients with chronic rhinosinusitis with nasal polyps. J Allergy Clin Immunol 2013; 132:584-92 e4.
54. Watanabe K, Jose P J, Rankin S M. Eotaxin-2 generation is differentially regulated by lipopolysaccharide and IL-4 in monocytes and macrophages. J Immunol 2002; 168:1911-8.
55. Wen T, Dellon E S, Moawad F J, Furuta G T, Aceves S S, Rothenberg M E. Transcriptome analysis of proton pump inhibitor-responsive esophageal eosinophilia reveals proton pump inhibitor-reversible allergic inflammation. J Allergy Clin Immunol 2015; 135:187-97.
56. Hamilos D L. Chronic rhinosinusitis: epidemiology and medical management. J Allergy Clin Immunol 2011; 128:693-707; quiz 8-9.
57. Fokkens W J, Lund V J, Mullol J, Bachert C, Alobid I, Baroody F, et al. European Position Paper on Rhinosinusitis and Nasal Polyps 2012. Rhinol Suppl 2012:3 p preceding table of contents, 1-298.
58. Dellon E S, Gonsalves N, Hirano I, Furuta G T, Liacouras C A, Katzka D A, et al. ACG clinical guideline: Evidenced based approach to the diagnosis and management of esophageal eosinophilia and eosinophilic esophagitis (EoE). Am J Gastroenterol 2013; 108:679-92; quiz 93.
59. Shin J M, Kim N. Pharmacokinetics and pharmacodynamics of the proton pump inhibitors. J Neurogastroenterol Motil 2013; 19:25-35.
60. Littner M R, Leung F W, Ballard E D, 2nd, Huang B, Samra N K, Lansoprazole Asthma Study G. Effects of 24 weeks of lansoprazole therapy on asthma symptoms, exacerbations, quality of life, and pulmonary function in adult asthmatic patients with acid reflux symptoms. Chest 2005; 128:1128-35.
61. Calabrese C, Fabbri A, Areni A, Scialpi C, Zahlane D, Di Febo G. Asthma and gastroesophageal reflux disease: effect of long-term pantoprazole therapy. World J Gastroenterol 2005; 11:7657-60.
62. American Lung Association Asthma Clinical Research C, Mastronarde J G, Anthonisen N R, Castro M, Holbrook J T, Leone F T, et al. Efficacy of esomeprazole for treatment of poorly controlled asthma. N Engl J Med 2009; 360:1487-99.
63. Modyanov N, Pestov N, Adams G, Crambert G, Tillekeratne M, Zhao H, et al. Nongastric H,K-ATPase: structure and functional properties. Ann N Y Acad Sci 2003; 986:183-7.
64. Modyanov N N, Petrukhin K E, Sverdlov V E, Grishin A V, Orlova M Y, Kostina M B, et al. The family of human Na,K-ATPase genes. ATP1AL1 gene is transcriptionally competent and probably encodes the related ion transport ATPase. FEBS Lett 1991; 278:91-4.
65. Swarts H G, Koenderink J B, Willems P H, De Pont J J. The non-gastric H,K-ATPase is oligomycin-sensitive and can function as an H+,NH4(+)-ATPase. J Biol Chem 2005; 280:33115-22.
66. Matsui M S, Petris M J, Niki Y, Karaman-Jurukovska N, Muizzuddin N, Ichihashi M, et al. Omeprazole, a gastric proton pump inhibitor, inhibits melanogenesis by blocking ATP7A trafficking. J Invest Dermatol 2015; 135:834-41.
67. Smith J J, Welsh M J. Fluid and electrolyte transport by cultured human airway epithelia. J Clin Invest 1993; 91:1590-7.
68. Shah V S, Meyerholz D K, Tang X X, Reznikov L, Abou Alaiwa M, Ernst S E, et al. Airway acidification initiates host defense abnormalities in cystic fibrosis mice. Science 2016; 351:503-7.
69. Van Den Berg J G, Aten J, Annink C, Ravesloot J H, Weber E, Weening J J. Interleukin-4 and -13 promote basolateral secretion of H(+) and cathepsin L by glomerular epithelial cells. Am J Physiol Renal Physiol 2002; 282:F26-33.
70. Danahay H, Atherton H, Jones G, Bridges R J, Poll C T. Interleukin-13 induces a hypersecretory ion transport phenotype in human bronchial epithelial cells. Am J Physiol Lung Cell Mol Physiol 2002; 282:L226-36.
71. Schulz E, Munzel T. Intracellular pH: a fundamental modulator of vascular function. Circulation 2011; 124:1806-7.
72. Yoshii K, Tajima F, Ishijima S, Sagami I. Changes in pH and NADPH regulate the DNA binding activity of neuronal PAS domain protein 2, a mammalian circadian transcription factor. Biochemistry 2015; 54:250-9.
73. Yuan Q, Campanella G S, Colvin R A, Hamilos D L, Jones K J, Mathew A, et al. Membrane-bound eotaxin-3 mediates eosinophil transepithelial migration in IL-4-stimulated epithelial cells. Eur J Immunol 2006; 36:2700-14.
74. Hunt J F, Fang K, Malik R, Snyder A, Malhotra N, Platts-Mills T A, et al. Endogenous airway acidification. Implications for asthma pathophysiology. Am J Respir Crit Care Med 2000; 161:694-9.
75. Kostikas K, Papatheodorou G, Ganas K, Psathakis K, Panagou P, Loukides S. pH in expired breath condensate of patients with inflammatory airway diseases. Am J Respir Crit Care Med 2002; 165:1364-70.
76. Cho D Y, Hajighasemi M, Hwang P H, Illek B, Fischer H. Proton secretion in freshly excised sinonasal mucosa from asthma and sinusitis patients. Am J Rhinol Allergy 2009; 23:e10-3.
77. Li X Q, Andersson T B, Ahlstrom M, Weidolf L. Comparison of inhibitory effects of the proton pump-inhibiting drugs omeprazole, esomeprazole, lansoprazole, pantoprazole, and rabeprazole on human cytochrome P450 activities. Drug Metab Dispos 2004; 32:821-7.
78. Howden C W, Meredith P A, Forrest J A, Reid J L. Oral pharmacokinetics of omeprazole. Eur J Clin Pharmacol 1984; 26:641-3.
79. Vaezi M F, Hagaman D D, Slaughter J C, Tanner S B, Duncavage J A, Allocco C T, et al. Proton pump inhibitor therapy improves symptoms in postnasal drainage. Gastroenterology 2010; 139:1887-93 el; quiz e11.
80. Pawar S, Lim H J, Gill M, Smith T L, Merati A, Toohill R J, et al. Treatment of postnasal drip with proton pump inhibitors: a prospective, randomized, placebo-controlled study. Am J Rhinol 2007; 21:695-701.
81. Min J Y Kern R C, Ocampo C J, Homma T, Conley D C, Shintani-Smith S, Huang H et al. Omeprazole has anti-inflammatory effects on Type-2 cytokine-stimulated human airway epithelial cells. J Allergy Clin Immunol 2015; 135:AB81.
82. Min J Y Kern R C, Ocampo C J, Stevens W W, Price C P E, Thompson C F et al. Proton pump inhibitos (PPIs) may modulate more than just reflux in chronic rhinosinusitis with nasal polyps. J Allergy Clin Immunol 2016; 137:AB28

1. A method of treating or ameliorating the symptoms of an eosinophilic inflammatory disease in a subject comprising inhibiting ATP12A expression and/or activity with the subject.

2. The method of claim 1, wherein the eosinophilic inflammatory disease is selected from the group consisting asthma, atopic dermatitis, eosinophilic esophagitis, chronic rhinosinusitis with nasal polyps (CRSwNP).

3. The method of claim 1, wherein inhibiting ATP12A expression and/or activity results in blunted IL-13-induction of eotaxin-3 mRNA, reduction in IL-13-induced epithelial cell production of eosinophil chemokines, suppression of the pathogenic effects of IL-4 and -13, and/or normalization of pH changes driven by type-2 cytokines.

4. The method of claim 1, wherein inhibiting ATP12A expression and/or activity comprises inhibiting expression of ATP12A.

5. The method of claim 4, wherein ATP12A expression is inhibited by inducing antisense inhibition, RNA interference, and or CRISPR/Cas.

6. The method of claim 1, wherein inhibiting ATP12A expression and/or activity comprises administering to the subject an inhibitor of ATP12A activity.

7. The method of claim 6, wherein the inhibitor is ATP12A specific.

8. The method of claim 6, wherein the inhibitor is a general H⁺/K⁺-ATPase inhibitor.

9. The method of claim 6, wherein the inhibitor is a small molecule, peptide, antibody, or antibody fragment.

10. The method of claim 9, wherein the inhibitor is a substituted benzimidazole compound.

11. The method of claim 10, wherein the substituted benzimidazole compound is selected from omeprazole, lansoprazole, dexlansoprazole, esomeprazole, pantoprazole, rabeprazole, and ilaprazole.

12. The method of claim 6, wherein the inhibitor is co-administered with an additional agent for treating or ameliorating the symptoms of an eosinophilic inflammatory disease.

13. The method of claim 12, wherein the inhibitor and additional agent are administered concurrently.

14. The method of claim 13, wherein the inhibitor and additional agent are co-formulated.

15. The method of claim 12, wherein the inhibitor an additional agent are administered serially.

16. A pharmaceutical composition comprising (a) an inhibitor of ATP12A expression and/or activity, (b) an additional agent for treating or ameliorating the symptoms of an eosinophilic inflammatory disease, and (c) a pharmaceutically-acceptable carrier.
